(19) United States
(12) Patent Application Publication
(10) Pub. No.: US 2019/0016856 A1
(43) Pub. Date: Jan. 17, 2019

(54) MULTI-ARMED POLYETHYLENE GLYCOL AND ACTIVE DERIVATIVE THEREOF

(71) Applicant: JenKem Technology Co., Ltd. (Beijing), Beijing (CN)

(72) Inventors: Zhen WEI, Beijing (CN); Meina LIN, Beijing (CN); Zhengang ZHU, Beijing (CN); Xuan ZHAO, Beijing (CN)

(21) Appl. No.: 16/133,248

(22) Filed: Sep. 17, 2018

Related U.S. Application Data
(63) Continuation of application No. PCT/CN2017/075599, filed on Mar. 3, 2017.

(30) Foreign Application Priority Data
Mar. 18, 2016 (CN) 201610158368.X

(51) Int. Cl. C08G 65/331; C08G 65/333; C08G 65/334; C08G 65/332

(52) U.S. Cl. CPC C08G 65/3312; C08G 65/33317; A61K 47/60; C08G 65/3322; C08G 65/3344

(57) ABSTRACT

Provided are a polyol glyceryl ether, and a multi-armed polyethylene glycol and the multi-armed polyethylene glycol active derivative prepared using the same. The multi-armed polyethylene glycol is formed by polymerizing ethylene oxide with the polyol glyceryl ether as an initiator, and has the structure of general formula II, wherein B is a polyol group, n is an integer between 3 and 22, PEG is the same or not the same —(OCH₂CH₂)ₘ—, and the average value of m is an integer between 3 and 250. The multi-armed polyethylene glycol has a relatively low poly-dispersity and a relatively high determined molecular weight. Also provided is a conjugate of the multi-armed polyethylene glycol active derivative and pharmaceutical molecules, a pharmaceutical composition comprising the conjugate, and a gel formed by the multi-armed polyethylene glycol active derivative. The gel can be used to prepare a sustained-release drug for prolonging the time of the drug action.

### CROSS-REFERENCE TO RELATED APPLICATION

This application is a continuation application of International patent application No. PCT/CN2017/075599, filed on Mar. 3, 2017, which claims the benefit and priority of Chinese patent application No. CN201610158368.X, filed on Mar. 18, 2016, each of which is incorporated herein by reference in its entirety and for all purposes.

### FIELD OF THE INVENTION

The present invention belongs to the technical field of polymer functional materials, and particularly relates to a multi-armed polyethylene glycol with a polyol glyceryl ether as a core, and an active functional group derivative, a drug conjugate, a gel material and a preparation method thereof, as well as use thereof in a pharmaceutical carrier, and a medical device gel.

### BACKGROUND OF THE INVENTION

Polyethylene glycol and its derivatives are widely used in biomedicine, pesticides, and medical materials due to their unique properties. Polyethylene glycol has a clear metabolic process in the human body and is a safe synthetic polymer material with no side effects. For example, when a protein, polypeptide or drug is conjugated with polyethylene glycol, it can effectively prolong its physiological half life and reduce its immunogenicity and toxicity. In clinical use, as a carrier for the production of pharmaceutical preparations, polyethylene glycol and its derivatives have been applied in many commercial drugs, and the direct bonding of polyethylene glycol to drug molecules has also been greatly developed in the last decade and has been applied in some approved drugs, such as conjugate of α-interferon and polyethylene glycol (PEGasys®), which shows a longer circulating half-life and better therapeutic effects. In addition, polyethylene glycol, as a safe synthetic polymer material with no side effects, has also been used in the preparation of novel medical devices. For example, new medical devices CoSeal of Baxter, and SprayGel and DuraSeal of Covidien, all use polyethylene glycol material.

Multi-armed polyethylene glycol is widely used as a novel polyethylene glycol material. Compared with linear polyethylene glycol, the multi-armed polyethylene glycol has a divergent and multi-branched structure, and multiple modifiable functional group sites in one molecule, which can realize loading of multiple drug molecules on a single molecule to improve the drug loading rate when it is applied in the field of drug modification. At the same time, since the end groups of the multi-armed polyethylene glycol product can be different functional groups, it is possible to realize the simultaneous linkage of two or even three kinds of drugs with one molecular system, thereby realizing treatment of a plurality of diseases by one molecular system. In addition, multi-armed polyethylene glycol with heterofunctional groups can also be used in the field of antibody-drug conjugates. Compared with linear linkers, multi-armed polyethylene glycol linker with heterofunctional groups can greatly increase the drug loading of a single antibody-drug conjugate molecule. In summary, multi-armed polyethylene glycol and its derivatives have broad application prospects.

The polyethylene glycol product with a narrow molecular weight distribution and low impurity content is an effective guarantee for the stability of a polyethylene glycol-modified bioactive drug molecule. As a polymeric polymer material used in the field of biomedicine, at present, there has been linear polyethylene glycol products with high quality and narrow molecular weight distribution, and it is a goal of those skilled in the art to produce a multi-armed polyethylene glycol product with high quality and narrow molecular weight distribution.

The multi-armed polyethylene glycol currently on the market has three, four, six and eight arms, etc. Wherein, three-armed and four-armed polyethylene glycols are formed by polymerization of ethylene oxide initiated by glycerol and pentaerythritol as central molecules, respectively. Since glycerol and pentaerythritol are single small molecules with high purity (>99%), the molecular weight distribution of the three-arm and four-arm polyethylene glycols produced by initiation with them is similar to that of linear polyethylene glycol, which can be reflected by a polydispersity coefficient of less than 1.08 in quality.

Six-armed and eight-armed polyethylene glycols were first formed by polymerization of ethylene oxide initiated by polyglycerol as a central molecule.

Polyglycerol is a liquid mixture, and the higher the degree of polymerization of polyglycerol, the more difficult it is to obtain a product with high purity. Therefore, the multi-armed polyethylene glycol synthesized by using polyglycerol as a central molecular initiator has a relatively broad molecular weight distribution and a polydispersity coefficient of more than 1.08. For example, the hexapolyglycerol required for the synthesis of eight-armed polyethylene glycol has a purity that is difficult to be higher than 85%, and the eight-armed polyethylene glycol synthesized by it as a central molecular initiator has a polydispersity coefficient much greater than 1.08 or even more than 1.10. The wide molecular weight distribution limits the pharmaceutical use of multi-armed polyethylene glycol with polyglycerol as a central molecular initiator.

Compared with polyglycerol, it is more likely to obtain an oligo-pentaerythritol product with a high purity, for example, dipentaerythritol and tripentaerythritol product may have a purity of about 95%. The multi-armed polyethylene glycol products synthesized by dipentaerythritol and tripentaerythritol as central molecular initiators have a reduced polydispersity coefficient and improved product quality. However, although dipentaerythritol and tripentaerythritol have much higher purity than polyglycerol, oligo-pentaerythritol is still a mixture, with a purity that is difficult to further increase. Therefore, the search for a novel single central molecule to replace polyglycerol and oligo-pentaerythritol has been a subject in the field of medical and pharmaceutical multi-armed polyethylene glycol. In addition, U.S. Pat. No. 6,587,376 describes a six-armed polyethylene glycol with sorbitol as a central molecular initiator. However, there are limitations for using saccharide molecules as central molecular initiators for requirements for more than six arms.

The present invention aims to overcome the defects of insufficient purity and wide molecular weight distribution of the multi-armed polyethylene glycol in the prior art, and provides a multi-armed polyethylene glycol with a novel structure, narrow molecular weight distribution and high purity, and a preparation method thereof, as well as an active derivative of the multi-armed polyethylene glycol, a gel formed therefrom, and a conjugate thereof with a drug molecule and use.

### SUMMARY OF THE INVENTION

In one aspect, the present invention provides a polyol glyceryl ether having a structure of formula I:

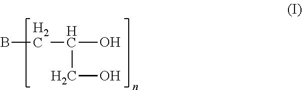

wherein, B is a polyol group, and n is an integer between 3 and 22.

Preferably, n is selected from the group consisting of 3, 4, 5, 6, 7, 8, 9, 10, 11, 12, 14, 16, 18, 20, 22; further preferably, n is selected from the group consisting of 3, 4, 5, 6, 7, 8, 10, 12; still further preferably, n is selected from the group consisting of 3, 4, 5, 6, 7, 8, 10; and most preferably, n is selected from the group consisting of 3, 4, 5, 6, 8.

Preferably, the polyol group B has a structure of formula B₁ or B₂:

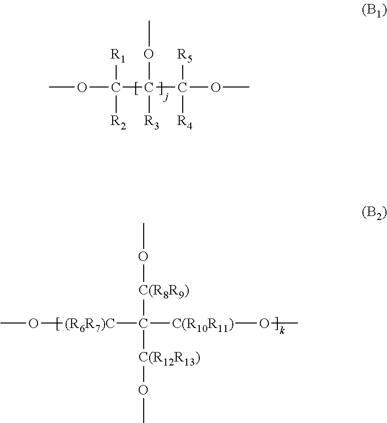

wherein, R₁-R₁₃ are independently selected from the group consisting of —H, C1-10 substituted or unsubstituted alkyl, substituted or unsubstituted aryl, substituted or unsubstituted aralkyl, substituted or unsubstituted aromatic or non-aromatic heterocyclic group;

preferably, R₁-R₁₃ are independently selected from the group consisting of —H, C1-5 substituted or unsubstituted alkyl, substituted or unsubstituted phenyl, substituted or unsubstituted benzyl, C3-18 substituted or unsubstituted aromatic or non-aromatic heterocyclic group;

further preferably, R₁-R₁₃ are independently selected from the group consisting of —H, methyl, ethyl, substituted or unsubstituted phenyl; and

j and k are independently selected from integers between 1 and 10, i.e., j and k are independently selected from the group consisting of 1, 2, 3, 4, 5, 6, 7, 8, 9, 10; preferably, j and k are independently selected from integers between 1 and 5, i.e., j and k are independently selected from the group consisting of 1, 2, 3, 4, 5; and most preferably, j and k are independently selected from integers between 1 and 4, i.e., j and k are independently selected from the group consisting of 1, 2, 3, 4.

In an embodiment of the present invention, the B has a structure of:

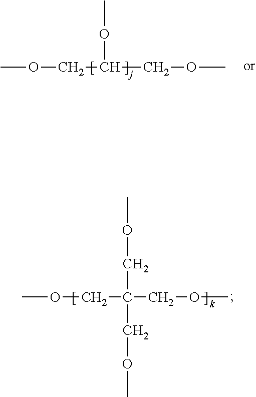

wherein, j and k are independently selected from integers between 1 and 10, i.e., j and k are independently selected from the group consisting of 1, 2, 3, 4, 5, 6, 7, 8, 9, 10; preferably, j and k are independently selected from integers between 1 and 5, i.e., j and k are independently selected from the group consisting of 1, 2, 3, 4, 5; and most preferably, j and k are independently selected from integers between 1 and 4, i.e., j and k are independently selected from the group consisting of 1, 2, 3, 4.

In a specific embodiment of the present invention, the polyol glyceryl ether includes, but is not limited to, glycerol triglyceryl ether (Ia1), butantetraol tetraglyceryl ether (Ia2), pentitol pentaglyceryl ether (Ia3), hexanehexol hexaglyceryl ether (Ia4), pentaerythritol tetraglyceryl ether (Ib1), dipentaerythritol hexaglyceryl ether (Ib2), and tripentaerythritol octaglyceryl ether (Ib3), the specific structure of which are as follows:

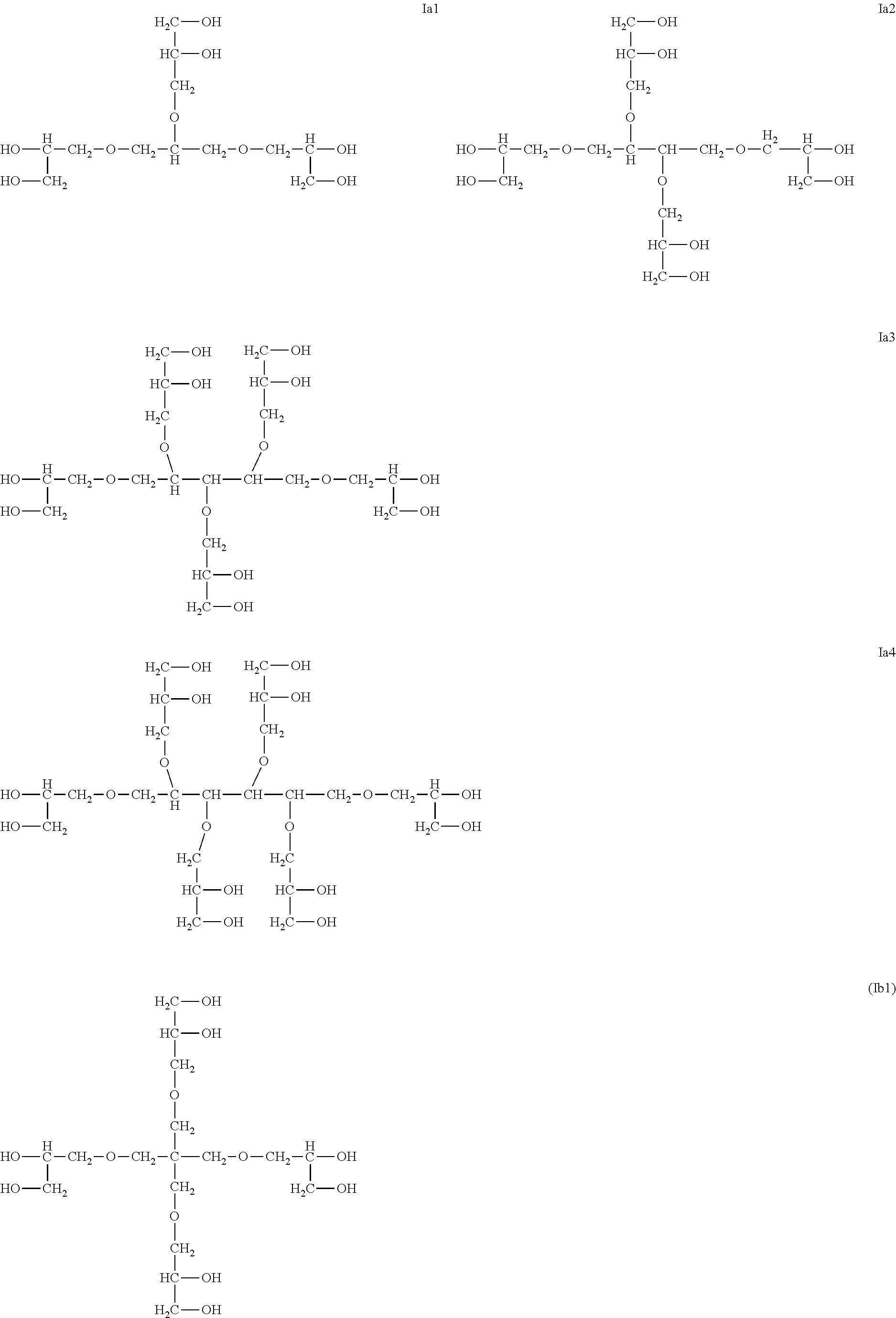 [SMILES: OCC(O)COCC(COCC(O)CO)OCC(O)CO ; OCC(O)COCC(OCC(O)CO)C(COCC(O)CO)OCC(O)CO ; OCC(O)COCC(OCC(O)CO)C(OCC(O)CO)C(COCC(O)CO)OCC(O)CO ; OCC(O)COCC(OCC(O)CO)C(OCC(O)CO)C(OCC(O)CO)C(COCC(O)CO)OCC(O)CO ; OCC(O)COCC(COCC(O)CO)(COCC(O)CO)COCC(O)CO]

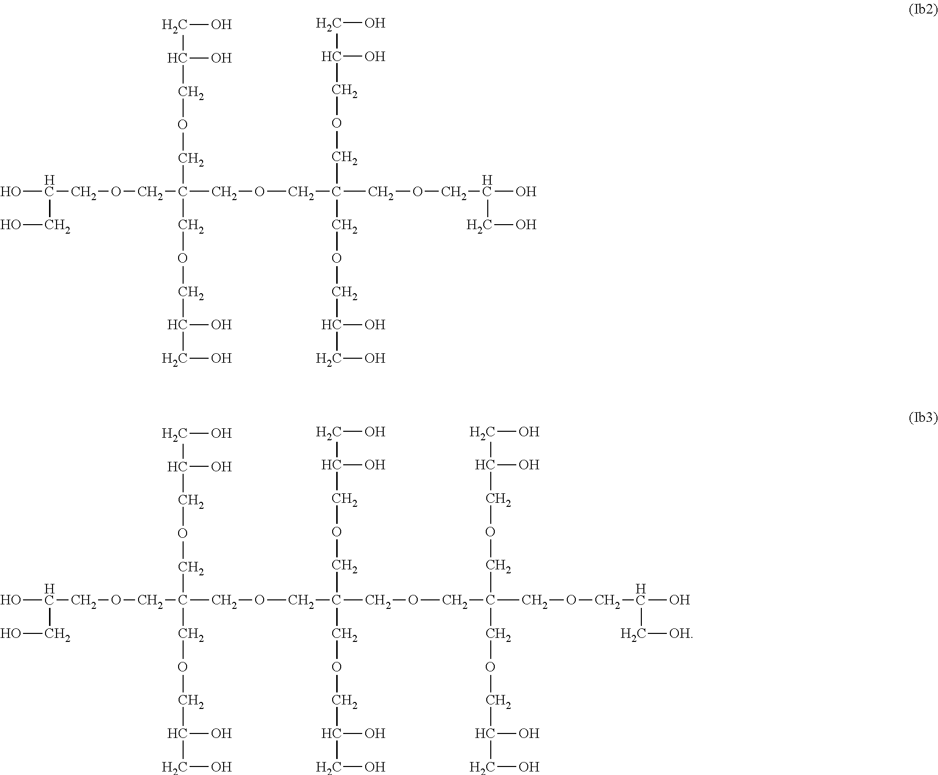 [SMILES: OCC(O)COCC(COCC(O)CO)(COCC(O)CO)COCC(COCC(O)CO)(COCC(O)CO)COCC(O)CO ; OCC(O)COCC(COCC(O)CO)(COCC(O)CO)COCC(COCC(O)CO)(COCC(O)CO)COCC(COCC(O)CO)(COCC(O)CO)COCC(O)CO]

In another aspect, the present invention provides a preparation method of the above-mentioned polyol glyceryl ether with high purity, the specific steps including: (1) catalyzing a reaction of

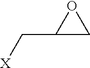

with a polyol using a catalyst 1 in a solvent to obtain a polyol glycidyl ether; and (2) catalyzing a hydrolysis reaction of the polyol glycidyl ether obtained in the step (1) using a catalyst 2 in a solvent to obtain the polyol glyceryl ether.

In the

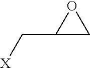

described in the step (1), X is selected from the group consisting of: F, Cl, Br, I,

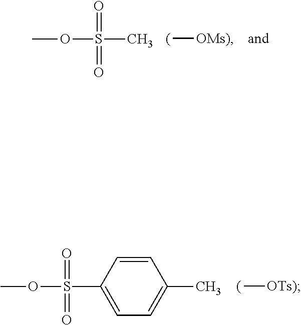

preferably Cl or Br.

The polyol described in the step (1) is an alcohol compound having 3 to 22 hydroxyl groups in the molecule and a structure of

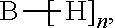

wherein, n is an integer between 3 and 22, B is a polyol group formed after the above-mentioned polyol loses hydroxy hydrogen, and H is a hydroxy hydrogen.

Preferably, n is selected from the group consisting of 3, 4, 5, 6, 7, 8, 9, 10, 11, 12, 14, 16, 18, 20, 22; further preferably, n is selected from the group consisting of 3, 4, 5, 6, 7, 8, 10, 12; still further preferably, n is selected from the group consisting of 3, 4, 5, 6, 7, 8, 10; and most preferably, n is selected from the group consisting of 3, 4, 5, 6, 8.

Preferably, the polyol group B has a structure of formula B₁ or B₂:

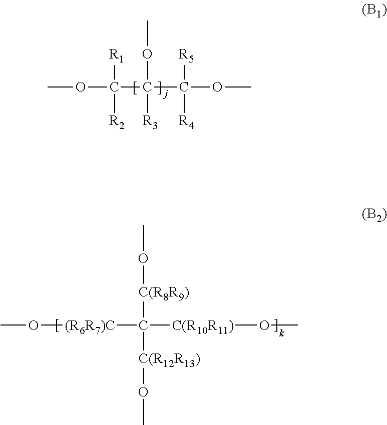

wherein, R₁-R₁₃ are independently selected from the group consisting of —H, C1-10 substituted or unsubstituted alkyl, substituted or unsubstituted aryl, substituted or unsubstituted aralkyl, substituted or unsubstituted aromatic or non-aromatic heterocyclic group;

preferably, R₁-R₁₃ are independently selected from the group consisting of —H, C1-5 substituted or unsubstituted alkyl, substituted or unsubstituted phenyl, substituted or unsubstituted benzyl, C3-18 substituted or unsubstituted aromatic or non-aromatic heterocyclic group;

further preferably, R₁-R₁₃ are independently selected from the group consisting of —H, methyl, ethyl, substituted or unsubstituted phenyl; and

j and k are independently selected from integers between 1 and 10, i.e., j and k are independently selected from the group consisting of 1, 2, 3, 4, 5, 6, 7, 8, 9, 10; preferably, j and k are independently selected from integers between 1 and 5, i.e., j and k are independently selected from the group consisting of 1, 2, 3, 4, 5; and most preferably, j and k are independently selected from integers between 1 and 4, i.e., j and k are independently selected from the group consisting of 1, 2, 3, 4.

In an embodiment of the present invention, the polyol has a structure of

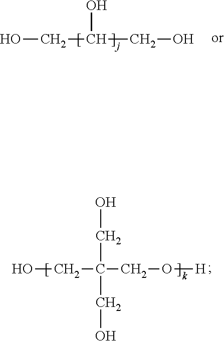

wherein j and k are independently selected from integers between 1 and 10, i.e., j and k are independently selected from the group consisting of 1, 2, 3, 4, 5, 6, 7, 8, 9, 10; preferably, j and k are independently selected from integers between 1 and 5, i.e., j and k are independently selected from the group consisting of 1, 2, 3, 4, 5; and most preferably, j and k are independently selected from integers between 1 and 4, i.e., j and k are independently selected from the group consisting of 1, 2, 3, 4.

In a specific embodiment of the present invention, the polyol includes, but is not limited to:

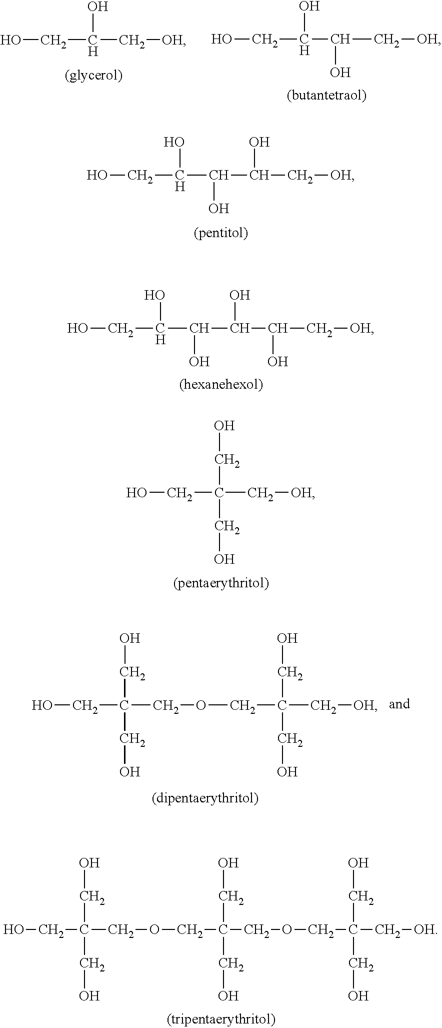

The catalyst 1 described in the step (1) is a base catalyst, and includes an organic base or an inorganic base, preferably is, but not limited to: pyridine, triethylamine, cesium carbonate, sodium carbonate, potassium carbonate, sodium bicarbonate, potassium bicarbonate, sodium hydroxide, potassium hydroxide, sodium alkoxide, or potassium alkoxide.

The catalyst 2 described in the step (2) is an acid catalyst or a base catalyst, preferably is, but not limited to: hydrochloric acid, sulfuric acid, phosphoric acid, trifluoroacetic acid, acetic acid, pyridine, triethylamine, cesium carbonate, sodium carbonate, potassium carbonate, sodium bicarbonate, potassium bicarbonate, sodium hydroxide, potassium hydroxide, sodium alkoxide, or potassium alkoxide.

The solvents described in the steps (1) and (2) include, but are not limited to: 1,4-dioxane, tetrahydrofuran, toluene, acetone, ethyl acetate, acetonitrile, N,N-dimethylformamide, dimethyl sulfoxide, and water.

Preferably, the above-mentioned preparation method of the polyol glyceryl ether with high purity includes the following specific steps: (1) adding a polyol, solvent and catalyst 1 to a reaction vessel, stirring, and dropwise adding halogenated or sulfonated propylene oxide to the mixture, controlling the reaction temperature to not exceed 35° C., after completion of the reaction, filtering, washing the filter residue, and collecting the filtrate and purifying to obtain a polyol glycidyl ester; and (2) dissolving the polyol glycidyl ester obtained in the step (1) in a solvent, adding a catalyst 2, reacting at 70-90° C. for 3-7 hours, after completion of the reaction, removing the solvent by rotary evaporation and purifying to obtain the polyol glyceryl ether.

Preferably, in the step (1), the molar ratio of monohydroxy group in the polyol to propylene oxide is 1:2 to 4.

Preferably, the purification step described in the step (1) includes: rotary evaporation, washing, extraction, molecular distillation, and column separation.

The general formula of the above reaction is as follows:

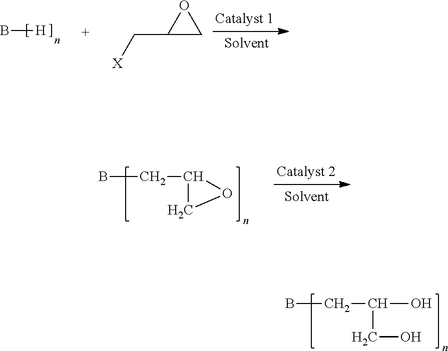

The polyol glyceryl ether synthesized by the above-mentioned preparation method has high purity of more than 99%. The preparation of high-purity polyol glyceryl ether lays a solid foundation for the synthesis of multi-armed polyethylene glycol with a high purity and narrow molecular weight distribution.

In another aspect, the present invention provides a novel multi-armed polyethylene glycol having a structure of formula II:

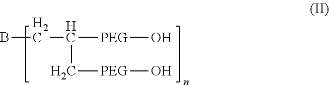

wherein, B is a polyol group, n is an integer between 3 and 22, PEG is the same or not the same —(OCH₂CH₂)ₘ—, and the average value of m is an integer between 3 and 250.

Preferably, n is selected from the group consisting of 3, 4, 5, 6, 7, 8, 9, 10, 11, 12, 14, 16, 18, 20, 22; further preferably, n is selected from the group consisting of 3, 4, 5, 6, 7, 8, 10, 12; still further preferably, n is selected from the group consisting of 3, 4, 5, 6, 7, 8, 10; and most preferably, n is selected from the group consisting of 3, 4, 5, 6, 8.

Preferably, the polyol group B has a structure of formula B₁ or B₂:

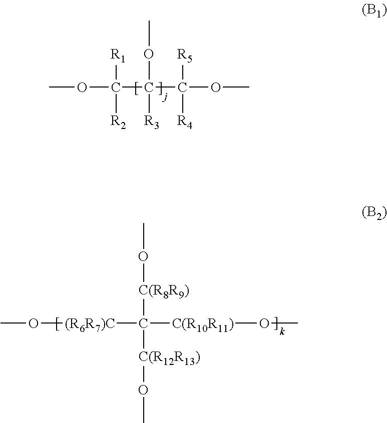

wherein, R₁-R₁₃ are independently selected from the group consisting of —H, C1-10 substituted or unsubstituted alkyl, substituted or unsubstituted aryl, substituted or unsubstituted aralkyl, substituted or unsubstituted aromatic or non-aromatic heterocyclic group;

preferably, R₁-R₁₃ are independently selected from the group consisting of —H, C1-5 substituted or unsubstituted alkyl, substituted or unsubstituted phenyl, substituted or unsubstituted benzyl, C3-18 substituted or unsubstituted aromatic or non-aromatic heterocyclic group;

further preferably, R₁-R₁₃ are independently selected from the group consisting of —H, methyl, ethyl, substituted or unsubstituted phenyl; and

j and k are independently selected from integers between 1 and 10, i.e., j and k are independently selected from the group consisting of 1, 2, 3, 4, 5, 6, 7, 8, 9, 10; preferably, j and k are independently selected from integers between 1 and 5, i.e., j and k are independently selected from the group consisting of 1, 2, 3, 4, 5; and most preferably, j and k are independently selected from integers between 1 and 4, i.e., j and k are independently selected from the group consisting of 1, 2, 3, 4.

Preferably, the average value of m is an integer between 10 and 200; further preferably, the average value of m is an integer between 20 and 150; still further preferably, the average value of m is an integer between 20 and 100; and most preferably, the average value of m is an integer between 20 and 80.

Preferably, the multi-armed polyethylene glycol has a number average molecular weight of 1,500 to 80,000, further preferably 5,000 to 60,000, still further preferably 10,000 to 50,000, and most preferably 10,000 to 30,000.

In an embodiment of the present invention, the B has a structure of:

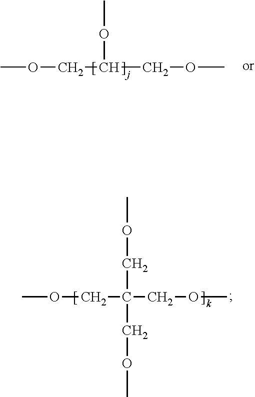

wherein, j and k are independently selected from integers between 1 and 10, i.e., j and k are independently selected from the group consisting of 1, 2, 3, 4, 5, 6, 7, 8, 9, 10; preferably, j and k are independently selected from integers between 1 and 5, i.e., j and k are independently selected from the group consisting of 1, 2, 3, 4, 5; and most preferably, j and k are independently selected from integers between 1 and 4, i.e., j and k are independently selected from the group consisting of 1, 2, 3, 4.

In a specific embodiment of the present invention, the multi-armed polyethylene glycol includes, but is not limited to, the following structures:

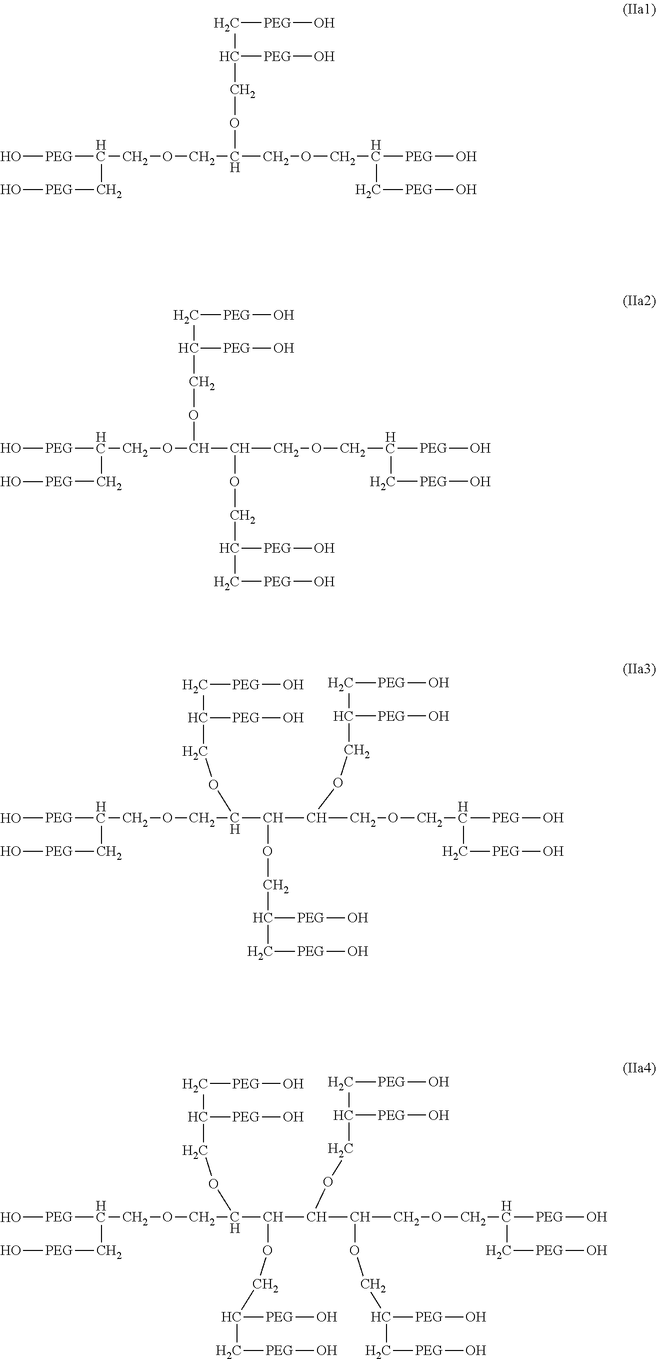

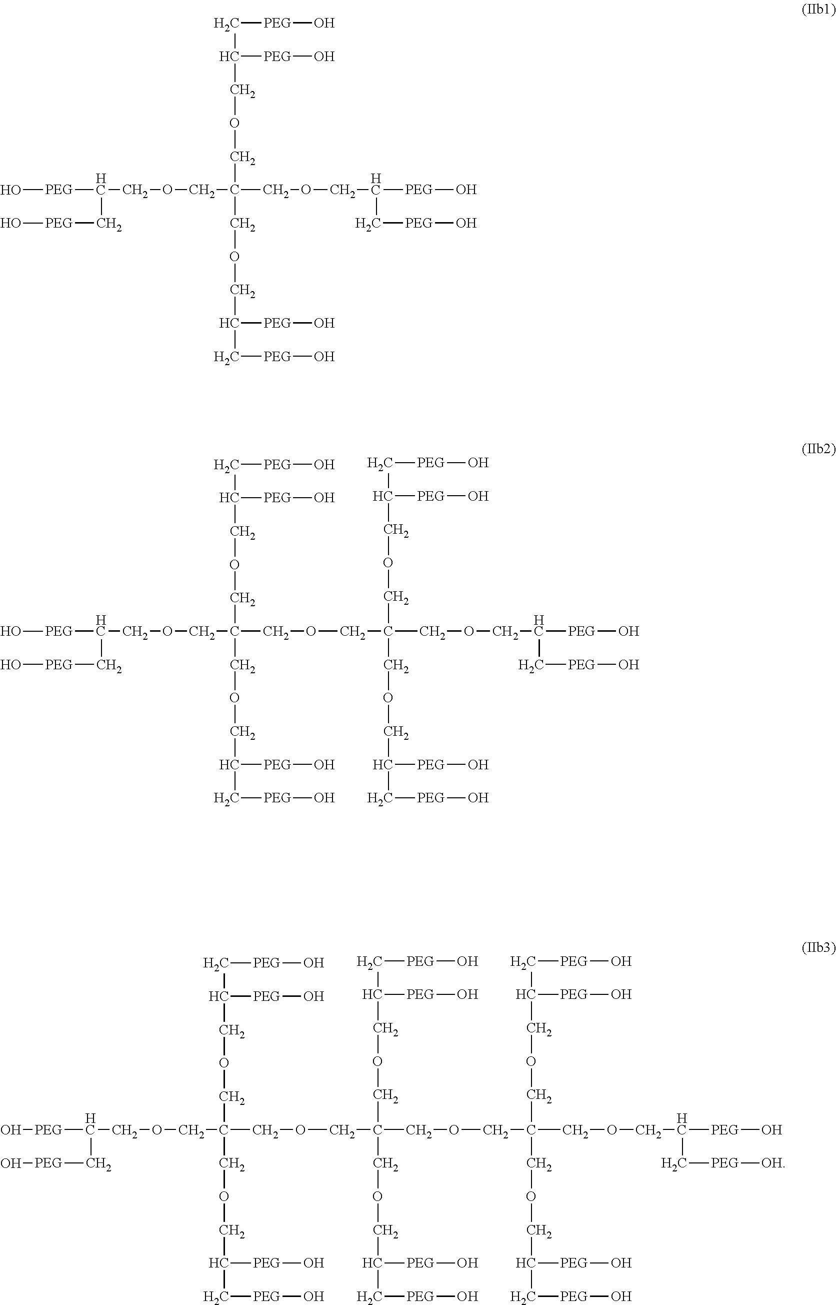

wherein, PEG is the same or not the same —(OCH₂CH₂)ₘ—, and the average value of m is an integer between 3 and 250; preferably, the average value of m is an integer between 10 and 200; further preferably, the average value of m is an integer between 20 and 150; still further preferably, the average value of m is an integer between 20 and 100; and most preferably, the average value of m is an integer between 20 and 80.

In another aspect, the present invention provides a preparation method of the above-mentioned multi-armed polyethylene glycol, which includes a step of polymerizing ethylene oxide with the above-mentioned polyol glyceryl ether as an initiator.

Preferably, the preparation method of the multi-armed polyethylene glycol includes the following specific steps: mixing the above-mentioned polyol glyceryl ether with a catalyst, heating, vacuuming, introducing ethylene oxide, reacting to obtain the multi-armed polyethylene glycol.

Preferably, the heating is heating to a temperature of 100-120° C.

Preferably, the vacuuming time is 1-3 hours.

The catalyst is selected from, but not limited to, potassium hydroxide, calcium hydroxide, calcium sulfate, and aluminum isopropoxide.

In another aspect, the present invention provides an active derivative of the above-mentioned novel multi-armed polyethylene glycol having a structure of formula III:

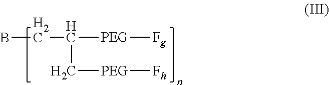

wherein, B is a polyol group, n is an integer between 3 and 22;

F_{g} and Fₕ are the same or not the same —Z—Y type structure;

Z is a linking group selected from the group consisting of —O(CH₂)ᵢ—, —O(CH₂)ᵢNH—, —O(CH₂)ᵢOCOO—, —O(CH₂)ᵢOCONH—, —O(CH₂)ᵢNHCOO—, —O(CH₂)ᵢNHCONH—, —OCO(CH₂)ᵢCOO—, —O(CH₂)ᵢCOO—, —O(CH₂)CONH— and —O(CH₂)ᵢNHCO(CH₂)ₑ—; i is an integer between 0 and 10, i.e., i is selected from 0, 1, 2, 3, 4, 5, 6, 7, 8, 9, and 10, and e is an integer between 1 and 10, i.e., e is selected from 1, 2, 3, 4, 5, 6, 7, 8, 9, and 10;

Y is a terminal active group, and

PEG is the same or not the same —(OCH₂CH₂)ₘ—, and the average value of m is an integer between 3 and 250.

Preferably, Y is selected from the group consisting of —H, —NH₂, —COCH═CH₂, —COC(CH₃)═CH₂,

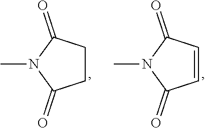

### —SH,

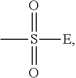

—CHO, —C≡CH, —PO₃H, —N₃, —CN, —CH═CH₂,

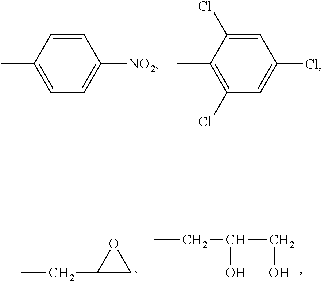

### —CH═Ch—COOH, —N═C═O,

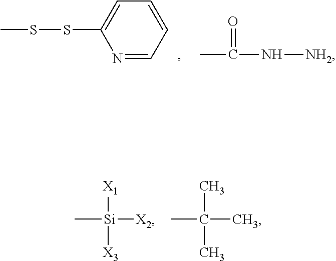

C1-6 alkyl and C1-6 alkoxy;

E is a C1-10 hydrocarbyl or a fluorine atom-containing C1-10 hydrocarbyl; and

X₁, X₂ and X₃ are the same or not the same C1-10 hydrocarbyl or C1-6 alkoxy. Preferably, n is selected from the group consisting of 3, 4, 5, 6, 7, 8, 9, 10, 11, 12, 14, 16, 18, 20, 22; further preferably, n is selected from the group consisting of 3, 4, 5, 6, 7, 8, 10, 12; still further preferably, n is selected from the group consisting of 3, 4, 5, 6, 7, 8, 10; and most preferably, n is selected from the group consisting of 3, 4, 5, 6, 8.

Preferably, the polyol group B has a structure of formula B₁ or B₂:

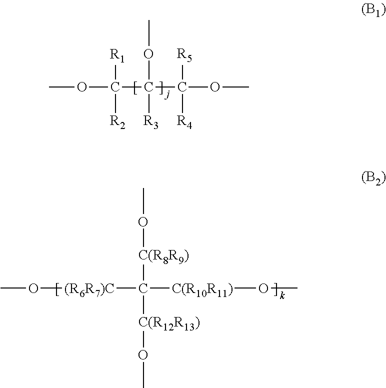

wherein, R₁-R₁₃ are independently selected from the group consisting of —H, C1-10 substituted or unsubstituted alkyl, substituted or unsubstituted aryl, substituted or unsubstituted aralkyl, substituted or unsubstituted aromatic or non-aromatic heterocyclic group.

Preferably, R₁-R₁₃ are independently selected from the group consisting of —H, C1-5 substituted or unsubstituted alkyl, substituted or unsubstituted phenyl, substituted or unsubstituted benzyl, C3-18 substituted or unsubstituted aromatic or non-aromatic heterocyclic group.

Further preferably, R₁-R₁₃ are independently selected from the group consisting of —H, methyl, ethyl, substituted or unsubstituted phenyl.

The j and k are independently selected from integers between 1 and 10, i.e., j and k are independently selected from the group consisting of 1, 2, 3, 4, 5, 6, 7, 8, 9, 10; preferably, j and k are independently selected from integers between 1 and 5, i.e., j and k are independently selected from the group consisting of 1, 2, 3, 4, 5; and most preferably, j and k are independently selected from integers between 1 and 4, i.e., j and k are independently selected from the group consisting of 1, 2, 3, 4.

Preferably, the average value of m is an integer between 10 and 200; further preferably, the average value of m is an integer between 20 and 150; still further preferably, the average value of m is an integer between 20 and 100; and most preferably, the average value of m is an integer between 20 and 80.

Preferably, i is an integer between 0 and 5, i.e., i is selected from 0, 1, 2, 3, 4, and 5; and further preferably, i is an integer between 0 to 3, i.e., i is selected from 0, 1, 2, and 3.

Preferably, e is an integer between 1 and 6, i.e., e is selected from 1, 2, 3, 4, 5, and 6; and further preferably, e is an integer between 1 and 3, i.e., e is selected from 1, 2, and 3.

Preferably, E is selected from the group consisting of methyl, ethyl, propyl, isopropyl, butyl, tert-butyl, pentyl, hexyl, heptyl, octyl, nonyl, decyl, vinyl, phenyl, benzyl, p-methylphenyl, trifluoromethyl, 2,2,2-trifluoroethyl, and 4-(trifluoromethoxy)phenyl; further preferably, E is selected from the group consisting of methyl, ethyl, propyl, butyl, vinyl, phenyl, benzyl, p-methylphenyl, and trifluoromethyl; still further preferably, E is selected from the group consisting of methyl, vinyl, and p-methylphenyl; and most preferably, E is methyl.

Preferably, X₁, X₂ and X₃ are independently selected from the group consisting of methyl, ethyl, propyl, isopropyl, butyl, tert-butyl, pentyl, hexyl, heptyl, octyl, nonyl, decyl, phenyl, benzyl, p-methylphenyl, methoxy, ethoxy, and propoxy; further preferably, X₁, X₂ and X₃ are independently is selected from the group consisting of methyl, ethyl, propyl, isopropyl, phenyl, benzyl, methoxy, and ethoxy; and most preferably, X₁, X₂ and X₃ are independently is selected from the group consisting of methyl, ethyl, methoxy, and ethoxy.

Preferably, Y is selected from the group consisting of —H, —NH₂, —COCH═CH₂, —COC(CH₃)═CH₂,

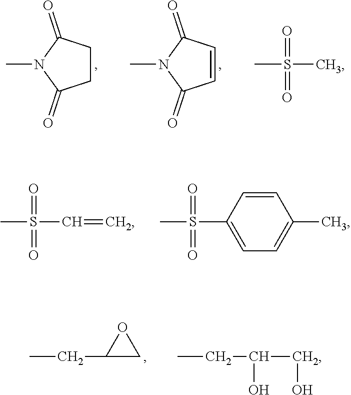

### —CH₂═CH—COOH, —N═C═O,

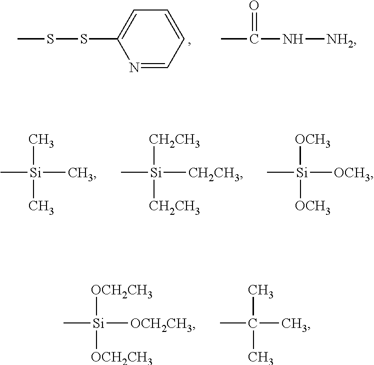

methyl, ethyl, propyl, isopropyl, methoxy, ethoxy, and propoxy; further preferably, Y is selected from the group consisting of —H, —NH₂, —COCH═CH₂,

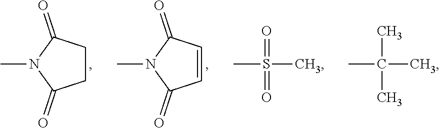

methyl, ethyl, methoxy, and ethoxy; and most preferably, Y is selected from the group consisting of: —H, —NH₂, —COCH═CH₂,

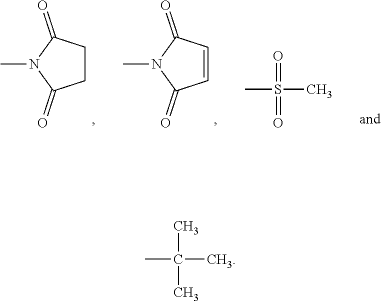

Preferably, the active derivative of the multi-armed polyethylene glycol has a number average molecular weight of 1,500 to 80,000, further preferably 5,000 to 60,000, still further preferably 10,000 to 50,000, and most preferably 10,000 to 30,000.

In an embodiment of the present invention, the B has a structure of:

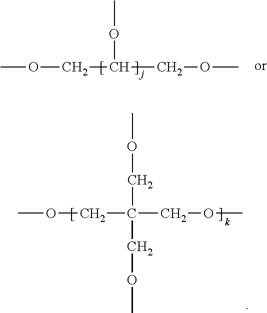

Wherein, j and k are independently selected from integers between 1 and 10, i.e., j and k are independently selected from the group consisting of 1, 2, 3, 4, 5, 6, 7, 8, 9, 10; preferably, j and k are independently selected from integers between 1 and 5, i.e., j and k are independently selected from the group consisting of 1, 2, 3, 4, 5; and most preferably, j and k are independently selected from integers between 1 and 4, i.e., j and k are independently selected from the group consisting of 1, 2, 3, 4.

In a specific embodiment of the present invention, the active derivative of the multi-armed polyethylene glycol includes, but is not limited to, the following structures:

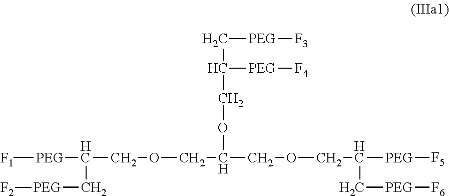

in formula IIIa1, F₁-F₆ are the same or not the same —Z—Y type structures,

Z₁-Z₆ are linking groups, and

Y₁-Y₆ are terminal active groups;

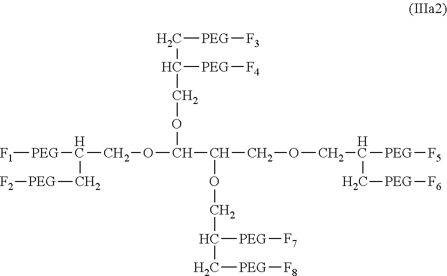

in formula IIIa2, F₁-F₈ are the same or not the same —Z—Y type structures,

Z₁-Z₈ are linking groups, and

Y₁-Y₈ are terminal active groups;

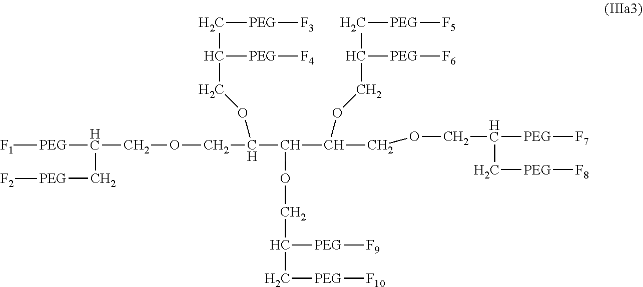

in formula IIIa3, F₁-F₁₀ are the same or not the same —Z—Y type structures,

Z₁-Z₁₀ are linking groups, and

Y₁-Y₁₀ are terminal active groups;

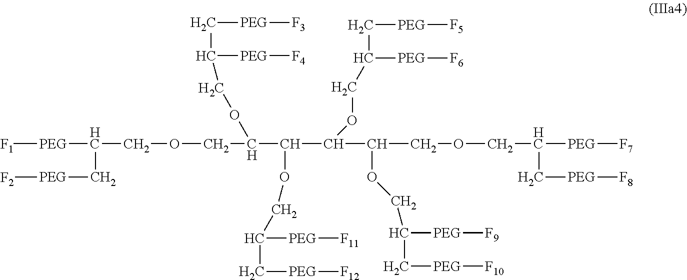

in formula IIIa4, F₁-F₁₂ are the same or not the same —Z—Y type structures,

Z₁-Z₁₂ are linking groups, and

Y₁-Y₁₂ are terminal active groups;

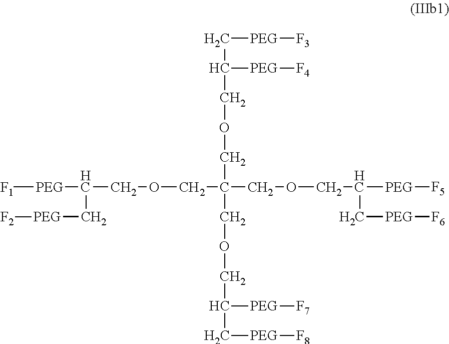

in formula IIIb1, F₁-F₈ are the same or not the same —Z—Y type structures,

Z₁-Z₈ are linking groups, and

Y₁-Y₈ are terminal active groups;

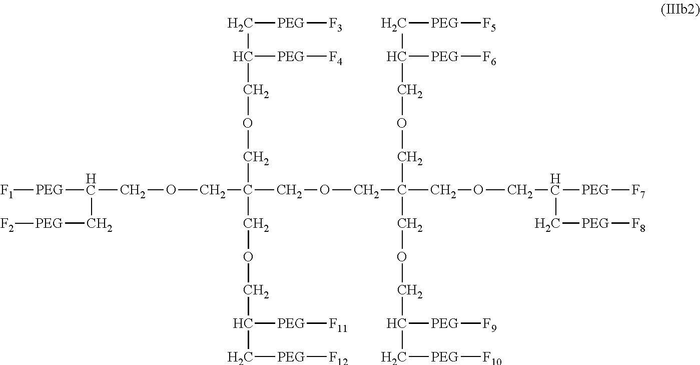

in formula IIIb2, F₁-F₁₂ are the same or not the same —Z—Y type structures,

Z₁-Z₁₂ are linking groups, and

Y₁-Y₁₂ are terminal active group;

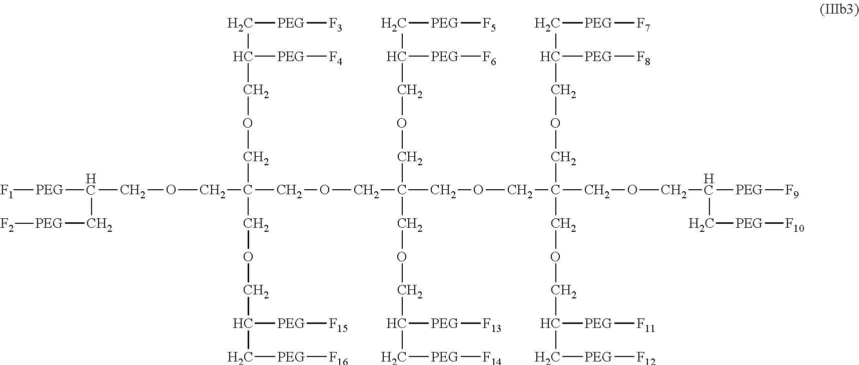

in formula IIIb3, F₁-F₁₆ are the same or not the same —Z—Y type structures,

Z₁-Z₁₆ are linking groups, and

Y₁-Y₁₆ are terminal active groups;

wherein,

the linking group is selected from the group consisting of —O(CH₂)ᵢ—, —O(CH₂)ᵢNH—, —O(CH₂)ᵢOCOO—, —O(CH₂)ᵢOCONH—, —O(CH₂)ᵢNHCOO—, —O(CH₂)ᵢNHCONH—, —OCO(CH₂)ᵢCOO—, —O(CH₂)ᵢCOO—, —O(CH₂)ᵢCONH— and —O(CH₂)ᵢNHCO(CH₂)ₑ—; i is an integer between 0 and 10, i.e., i is selected from 0, 1, 2, 3, 4, 5, 6, 7, 8, 9, and 10; preferably, i is an integer between 0 and 5, i.e., i is selected from 0, 1, 2, 3, 4, and 5; further preferably, i is an integer between 0 and 3, i.e., i is selected from 0, 1, 2, and 3; and e is an integer between 1 and 10, i.e., e is selected from 1, 2, 3, 4, 5, 6, 7, 8, 9, and 10; preferably, e is an integer between 1 and 6, i.e., e is selected from 1, 2, 3, 4, 5, and 6; further preferably, e is an integer between 1 and 3, i.e., e is selected from 1, 2, and 3;

the terminal active group is selected from the group consisting of —H, —NH₂, —COCH═CH₂, —COC(CH₃)═CH₂,

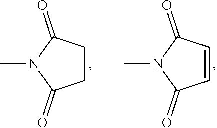

### —SH,

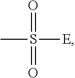

—CHO, —C≡CH, —PO₃H, —N₃, —CN, —CH═CH₂,

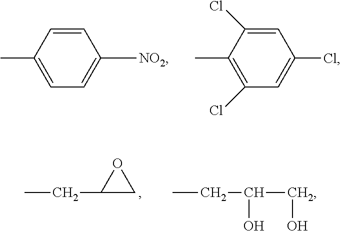

### —CH═CH—COOH, —N═C═O,

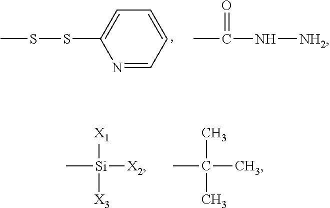

C1-6 alkyl and C1-6 alkoxy; preferably, the terminal active group is selected from the group consisting of —H, —NH₂, —COCH═CH₂, —COC(CH₃)═CH₂,

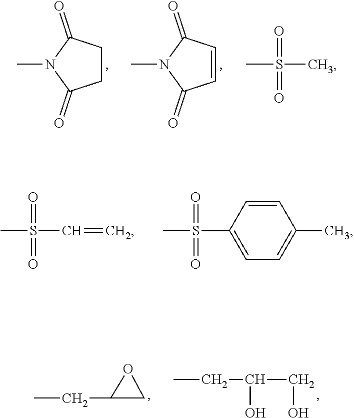

### —CH═CH—COOH, —N═C═O,

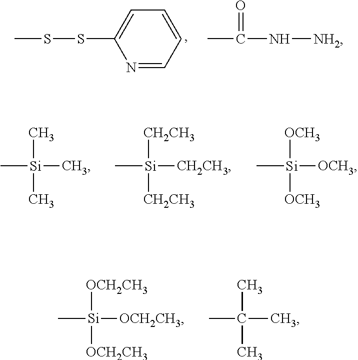

methyl, ethyl, propyl, isopropyl, methoxy, ethoxy, and propoxy; further preferably, the terminal active group is selected from the group consisting of —H, —NH₂, —COCH═CH₂,

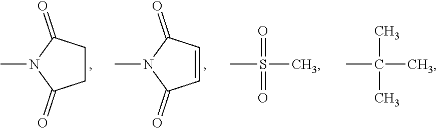

methyl, ethyl, methoxy, and ethoxy; and most preferably, the terminal active group is selected from the group consisting of: —H, —NH₂, —COCH═CH₂,

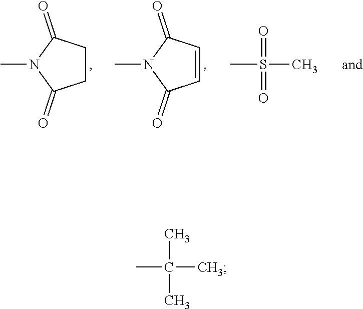

E is a C1-10 hydrocarbyl or a fluorine atom-containing C1-10 hydrocarbyl; preferably, E is selected from the group consisting of methyl, ethyl, propyl, isopropyl, butyl, tert-butyl, pentyl, hexyl, heptyl, octyl, nonyl, decyl, vinyl, phenyl, benzyl, p-methylphenyl, trifluoromethyl, 2,2,2-trifluoroethyl, and 4-(trifluoromethoxy)phenyl; further preferably, E is selected from the group consisting of methyl, ethyl, propyl, butyl, vinyl, phenyl, benzyl, p-methylphenyl, and trifluoromethyl; still further preferably, E is selected from the group consisting of methyl, vinyl, and p-methylphenyl; and most preferably, E is methyl;

X₁, X₂ and X₃ are the same or not the same C1-10 hydrocarbyl or C1-6 alkoxy; preferably, X₁, X₂ and X₃ are independently selected from the group consisting of methyl, ethyl, propyl, isopropyl, butyl, tert-butyl, pentyl, hexyl, heptyl, octyl, nonyl, decyl, phenyl, benzyl, p-methylphenyl, methoxy, ethoxy, and propoxy; further preferably, X₁, X₂ and X₃ are independently selected from the group consisting of methyl, ethyl, propyl, isopropyl, phenyl, benzyl, methoxy, and ethoxy; and most preferably, X₁, X₂ and X₃ are independently selected from the group consisting of methyl, ethyl, methoxy, and ethoxy; and

PEG is the same or not the same —(OCH₂CH₂)ₘ—, and the average value of m is an integer between 3 and 250; preferably, the average value of m is an integer between 10 and 200; further preferably, the average value of m is an integer between 20 and 150; still further preferably, the average value of m is an integer between 20 and 100; and most preferably an integer between 20 and 80.

In an embodiment of the present invention, in the active derivative of the multi-armed polyethylene glycol, the F_{g} and Fₕ are different —Z—Y type structures;

wherein, F₁-Fₜ is a —Z₁—Y₁ type structure;

Fₜ₊₁-F₂ₙ is a —Z₂—Y₂ type structure;

t is an integer and 1≤t≤2n−1; preferably, t is an integer between 1 and 5, i.e., t is selected from 1, 2, 3, 4, and 5; further preferably, t is an integer between 1 and 3, i.e., t is selected from 1, 2, and 3; and most preferably, t is 1 or 2;

Z₁ is a linking group selected from the group consisting of —O(CH₂)ᵢOCOO—, —O(CH₂)ᵢOCONH—, —OCO(CH₂)ᵢCOO—, —O(CH₂)ᵢCOO— and —O(CH₂)ᵢCONH—; i is an integer between 0 and 10; preferably, i is an integer between 0 and 5, i.e., i is selected from 0, 1, 2, 3, 4, and 5; and further preferably, i is an integer between 0 to 3, i.e., i is selected from 0, 1, 2, and 3;

Z₂ is a linking group selected from the group consisting of —O(CH₂)_{i′}—, —O(CH₂)_{i′}NH—, —O(CH₂)_{i′}NHCOO—, —O(CH₂)_{i′}NHCONH— and —O(CH₂)_{i′}NHCO(CH₂)ₑ—; i′ is an integer between 0 and 10; preferably, i′ is an integer between 0 and 5, i.e., i′ is selected from 0, 1, 2, 3, 4, and 5; further preferably, i′ is an integer between 0 and 3, i.e., i′ is selected from 0, 1, 2, and 3; and e is an integer between 1 and 10, i.e., e is selected from 1, 2, 3, 4, 5, 6, 7, 8, 9, and 10; preferably, e is an integer between 1 and 6, i.e., e is selected from 1, 2, 3, 4, 5, and 6; and further preferably, e is an integer between 1 and 3, i.e., e is selected from 1, 2, and 3;

Y is a terminal active group selected from the group consisting of —H, —NH₂, —COCH═CH₂, —COC(CH₃)═CH₂,

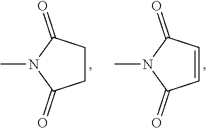

### —SH,

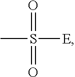

### —CHO, —C≡CH,

—PO₃H, —N₃, —CN, —CH═CH₂,

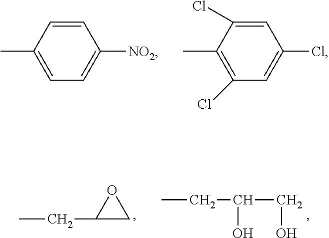

### —CH═CH—COOH, —N═C═O,

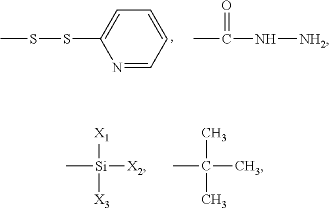

C1-6 alkyl and C1-6 alkoxy; preferably, Y is selected from the group consisting of —H, —NH₂, —COCH═CH₂, —COC(CH₃)═CH₂,

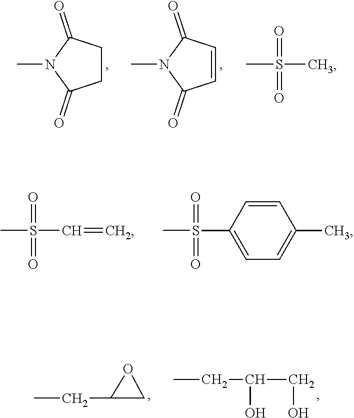

### —CH═CH—COOH, —N═C═O,

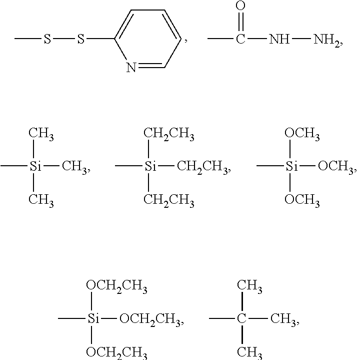

methyl, ethyl, propyl, isopropyl, methoxy, ethoxy, and propoxy; further preferably, Y is selected from the group consisting of —H, —NH₂, —COCH═CH₂,

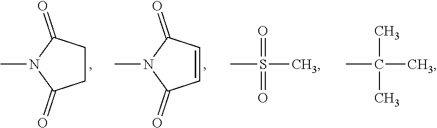

methyl, ethyl, methoxy, and ethoxy; and most preferably, Y is selected from the group consisting of: —H, —NH₂, —COCH═CH₂,

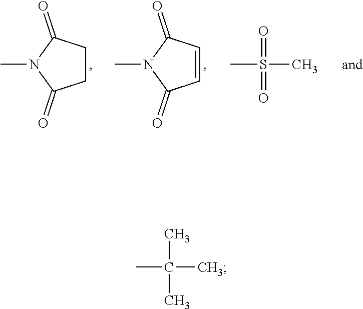

E is a C1-10 hydrocarbyl or a fluorine atom-containing C1-10 hydrocarbyl; preferably, E is selected from the group consisting of methyl, ethyl, propyl, isopropyl, butyl, tert-butyl, pentyl, hexyl, heptyl, octyl, nonyl, decyl, vinyl, phenyl, benzyl, p-methylphenyl, trifluoromethyl, 2,2,2-trifluoroethyl, and 4-(trifluoromethoxy)phenyl; further preferably, E is selected from the group consisting of methyl, ethyl, propyl, butyl, vinyl, phenyl, benzyl, p-methylphenyl, and trifluoromethyl; still further preferably, E is selected from the group consisting of methyl, vinyl, and p-methylphenyl; and most preferably, E is methyl; and

X₁, X₂ and X₃ are the same or not the same C1-10 hydrocarbyl or C1-6 alkoxy; preferably, X₁, X₂ and X₃ are independently selected from the group consisting of methyl, ethyl, propyl, isopropyl, butyl, tert-butyl, pentyl, hexyl, heptyl, octyl, nonyl, decyl, phenyl, benzyl, p-methylphenyl, methoxy, ethoxy, and propoxy; further preferably, X₁, X₂ and X₃ are independently selected from the group consisting of methyl, ethyl, propyl, isopropyl, phenyl, benzyl, methoxy, and ethoxy; and most preferably, X₁, X₂ and X₃ are independently selected from the group consisting of methyl, ethyl, methoxy, and ethoxy.

In a specific embodiment of the present invention, the active derivative of the multi-armed polyethylene glycol is a six-armed polyethylene glycol-monoacid derivative having a structure of the following formula IIIa1-a1:

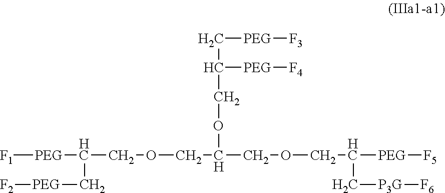

wherein, F₁ is a —Z—Y type structure different from F₂, F₃, F₄, F₅ and F₆;

F₁ is a —Z₁—Y₁ type structure;

F₂, F₃, F₄, F₅ and F₆ are —Z₂—Y₂ type structures;

Z₁ is a linking group selected from the group consisting of —O(CH₂)ᵢOCOO—, —O(CH₂)ᵢOCONH—, —OCO(CH₂)ᵢCOO—, —O(CH₂)ᵢCOO— and —O(CH₂)ᵢCONH—; i is an integer between 0 and 10; preferably, i is an integer between 0 and 5, i.e., i is selected from 0, 1, 2, 3, 4, and 5; and further preferably, i is an integer between 0 and 3, i.e., i is selected from 0, 1, 2, and 3;

Z₂ is a linking group selected from the group consisting of —O(CH₂)_{i′}—, —O(CH₂)_{i′}NH—, —O(CH₂)ᵢNHCOO—, —O(CH₂)_{i′}NHCONH— and —O(CH₂)_{i′}NHCO(CH₂)ₑ—; i′ is an integer between 0 and 10; preferably, i′ is an integer between 0 and 5, i.e., i′ is selected from 0, 1, 2, 3, 4, and 5; and further preferably, i′ is an integer between 0 and 3, i′ is selected from 0, 1, 2, and 3; and e is an integer between 1 and 10, i.e., e is selected from 1, 2, 3, 4, 5, 6, 7, 8, 9, and 10; preferably, e is an integer between 1 and 6, i.e., e is selected from 1, 2, 3, 4, 5, and 6; and further preferably, e is an integer between 1 and 3, i.e., e is selected from 1, 2, and 3;

Y is a terminal active group selected from the group consisting of —H, —NH₂, —COCH═CH₂, —COC(CH₃)═CH₂,

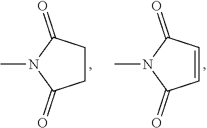

### —SH,

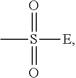

—CHO, —C≡CH, —PO₃H, —N₃, —CN, —CH═CH₂,

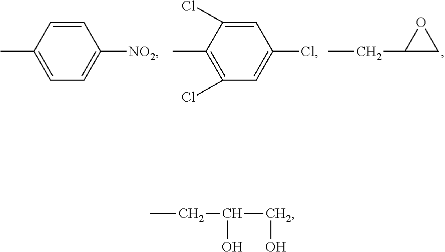

### —CH═CH—COOH, —N═C═O,

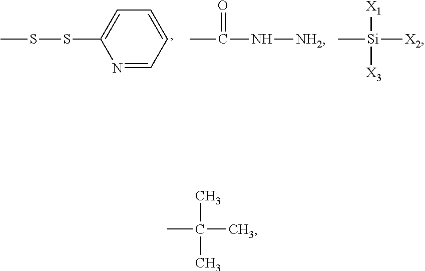

C1-6 alkyl and C1-6 alkoxy; preferably, Y is selected from the group consisting of —H, —NH₂, —COCH═CH₂, —COC(CH₃)═CH₂,

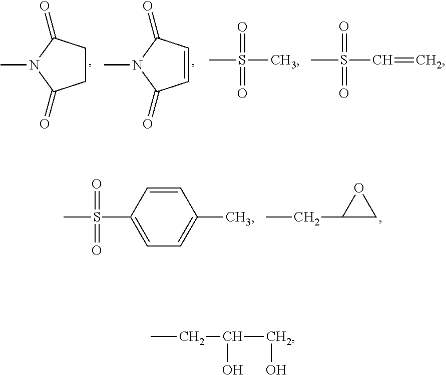

### —CH═CH—COOH, —N═C═O,

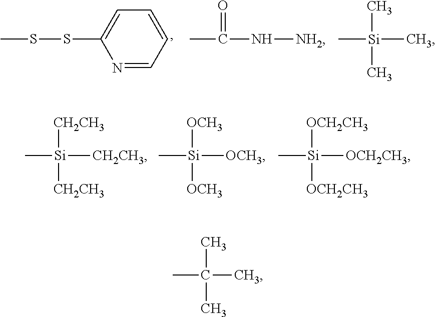

methyl, ethyl, propyl, isopropyl, methoxy, ethoxy, and propoxy; further preferably, Y is selected from the group consisting of —H, —NH₂, —COCH═CH₂,

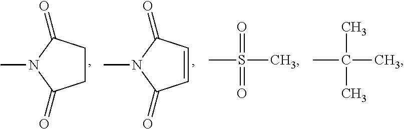

methyl, ethyl, methoxy, and ethoxy; and most preferably, Y is selected from the group consisting of: —H, —NH₂, —COCH═CH₂,

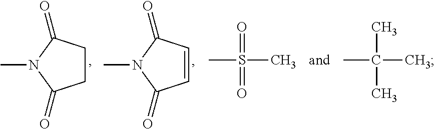

E is a C1-10 hydrocarbyl or a fluorine atom-containing C1-10 hydrocarbyl; preferably, E is selected from the group consisting of methyl, ethyl, propyl, isopropyl, butyl, tert-butyl, pentyl, hexyl, heptyl, octyl, nonyl, decyl, vinyl, phenyl, benzyl, p-methylphenyl, trifluoromethyl, 2,2,2-trifluoroethyl, and 4-(trifluoromethoxy)phenyl; further preferably, E is selected from the group consisting of methyl, ethyl, propyl, butyl, vinyl, phenyl, benzyl, p-methylphenyl, and trifluoromethyl; still further preferably, E is selected from the group consisting of methyl, vinyl, and p-methylphenyl; and most preferably, E is methyl;

X₁, X₂ and X₃ are the same or not the same C1-10 hydrocarbyl or C1-6 alkoxy; preferably, X₁, X₂, and X₃ are independently selected from the group consisting of methyl, ethyl, propyl, isopropyl, butyl, tert-butyl, pentyl, hexyl, heptyl, octyl, nonyl, decyl, phenyl, benzyl, p-methylphenyl, methoxy, ethoxy, and propoxy; further preferably, X₁, X₂, and X₃ are independently selected from the group consisting of methyl, ethyl, propyl, isopropyl, phenyl, benzyl, methoxy, and ethoxy; and most preferably, X₁, X₂, and X₃ are independently selected from the group consisting of methyl, ethyl, methoxy, and ethoxy; and

PEG is the same or not the same —(OCH₂CH₂)ₘ—, and the average value of m is an integer between 3 and 250; preferably, the average value of m is an integer between 10 and 200; further preferably, the average value of m is an integer between 20 and 150; still further preferably, the average value of m is an integer between 20 and 100; and most preferably an integer between 20 and 80.

In a specific embodiment of the present invention, the active derivative of the multi-armed polyethylene glycol is an eight-armed polyethylene glycol-monoacid derivative having a structure of the following formula IIIb1-a1:

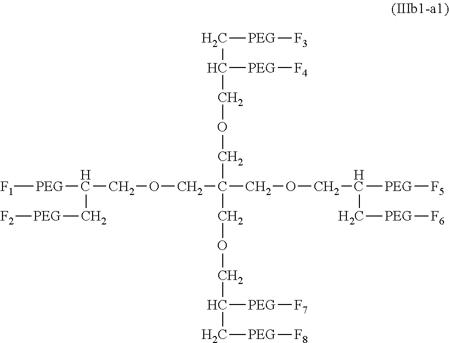

wherein, F₁ is a —Z—Y type structure different from F₂, F₃, F₄, F₅, F₆, F₇ and F₈;

F₁ is a —Z₁—Y₁ type structure;

F₂, F₃, F₄, F₅, F₆, F₇ and F₈ are —Z₂—Y₂ type structures;

Z₁ is a linking group selected from the group consisting of —O(CH₂)ᵢOCOO—, —O(CH₂)ᵢOCONH—, —OCO(CH₂)ᵢCOO—, —O(CH₂)ᵢCOO— and —O(CH₂)ᵢCONH—; i is an integer between 0 and 10; preferably, i is an integer between 0 and 5, i.e., i is selected from 0, 1, 2, 3, 4, and 5; and further preferably, i is an integer between 0 and 3, i.e., i is selected from 0, 1, 2, and 3;

Z₂ is a linking group selected from the group consisting of —O(CH₂)_{i′}—, —O(CH₂)_{i′}NH—, —O(CH₂)_{i′}NHCOO—, —O(CH₂)_{i′}NHCONH— and —O(CH₂)_{i′}NHCO(CH₂)ₑ—; i′ is an integer between 0 and 10; preferably, i′ is an integer between 0 and 5, i.e., i′ is selected from 0, 1, 2, 3, 4, and 5; and further preferably, i′ is an integer between 0 and 3, i′ is selected from 0, 1, 2, and 3; and e is an integer between 1 and 10, i.e., e is selected from 1, 2, 3, 4, 5, 6, 7, 8, 9, and 10; preferably, e is an integer between 1 and 6, i.e., e is selected from 1, 2, 3, 4, 5, and 6; and further preferably, e is an integer between 1 and 3, i.e., e is selected from 1, 2, and 3;

Y is a terminal active group selected from the group consisting of —H, —NH₂, —COCH═CH₂, —COC(CH₃)═CH₂,

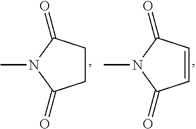

### —SH,

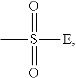

—CHO, —C≡CH, —PO₃H, —N₃, —CN, —CH═CH₂,

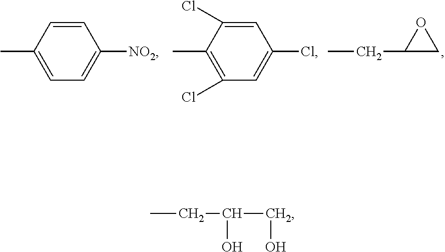

### —CH═CH—COOH, —N═C═O,

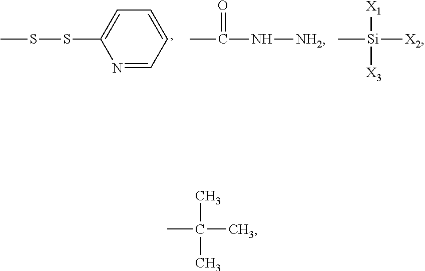

C1-6 alkyl and C1-6 alkoxy; preferably, Y is selected from the group consisting of —H, —NH₂, —COCH═CH₂, —COC(CH₃)═CH₂,

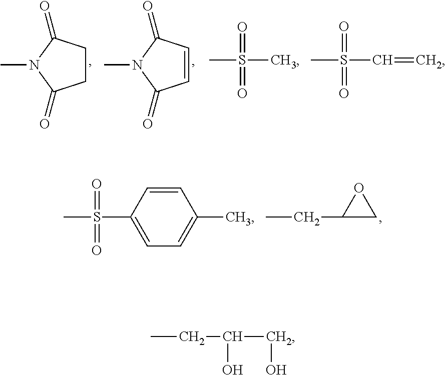

### —CH═Ch—COOH, —N═C═O,

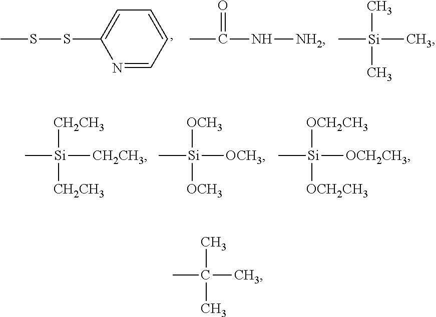

methyl, ethyl, propyl, isopropyl, methoxy, ethoxy, and propoxy; further preferably, Y is selected from
the group consisting of —H, —NH₂, —COCH═CH₂,

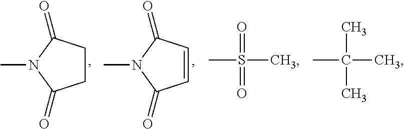

methyl, ethyl, methoxy, and ethoxy; and most preferably, Y is selected from the group consisting of: —H, —NH₂, —COCH═CH₂,

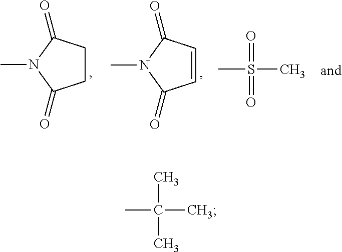

E is a C1-10 hydrocarbyl or a fluorine atom-containing C1-10 hydrocarbyl; preferably, E is selected from the group consisting of methyl, ethyl, propyl, isopropyl, butyl, tert-butyl, pentyl, hexyl, heptyl, octyl, nonyl, decyl, vinyl, phenyl, benzyl, p-methylphenyl, trifluoromethyl, 2,2,2-trifluoroethyl, and 4-(trifluoromethoxy)phenyl; further preferably, E is selected from the group consisting of methyl, ethyl, propyl, butyl, vinyl, phenyl, benzyl, p-methylphenyl, and trifluoromethyl; still further preferably, E is selected from the group consisting of methyl, vinyl, and p-methylphenyl; and most preferably, E is methyl;

X₁, X₂ and X₃ are the same or not the same C1-10 hydrocarbyl or C1-6 alkoxy; preferably, X₁, X₂ and X₃ are independently selected from the group consisting of methyl, ethyl, propyl, isopropyl, butyl, tert-butyl, pentyl, hexyl, heptyl, octyl, nonyl, decyl, phenyl, benzyl, p-methylphenyl, methoxy, ethoxy, and propoxy; further preferably, X₁, X₂ and X₃ are independently selected from the group consisting of methyl, ethyl, propyl, isopropyl, phenyl, benzyl, methoxy, and ethoxy; and most preferably, X₁, X₂ and X₃ are independently selected from the group consisting of methyl, ethyl, methoxy, and ethoxy; and

PEG is the same or not the same —(OCH₂CH₂)ₘ—, and the average value of m is an integer between 3 and 250; preferably, the average value of m is an integer between 10 and 200; further preferably, the average value of m is an integer between 20 and 150; still further preferably, the average value of m is an integer between 20 and 100; and most preferably an integer between 20 and 80.

In a specific embodiment of the present invention, the active derivative of the multi-armed polyethylene glycol is an eight-armed polyethylene glycol-diacid derivative having a structure of the following formula IIIb1-a2:

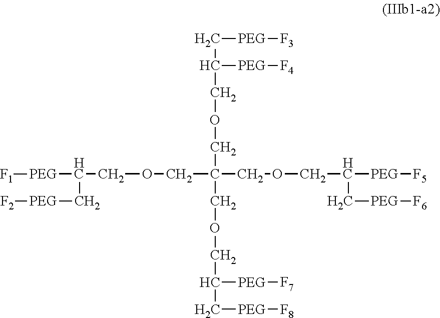

wherein, F₁ and F₂ are —Z—Y type structures different from F₃, F₄, F₅, F₆, F₇ and F₈;

F₁ and F₂ are —Z₁—Y₁ type structures;

F₃, F₄, F₅, F₆, F₇ and F₈ are —Z₂—Y₂ type structures;

Z₁ is a linking group selected from the group consisting of —O(CH₂)ᵢOCOO—, —O(CH₂)ᵢOCONH—, —OCO(CH₂)ᵢCOO—, —O(CH₂)ᵢCOO— and —O(CH₂)ᵢCONH—; i is an integer between 0 and 10; preferably, i is an integer between 0 and 5, i.e., i is selected from 0, 1, 2, 3, 4, and 5; and further preferably, i is an integer between 0 and 3, i.e., i is selected from 0, 1, 2, and 3;

Z₂ is a linking group selected from the group consisting of —O(CH₂)_{i′}—, —O(CH₂)_{i′}NH—, —O(CH₂)_{i′}NHCOO—, —O(CH₂)_{i′}NHCONH— and —O(CH₂)_{i′}NHCO(CH₂)ₑ—; i′ is an integer between 0 and 10; preferably, i′ is an integer between 0 and 5, i.e., i′ is selected from 0, 1, 2, 3, 4, and 5; and further preferably, i′ is an integer between 0 and 3, i′ is selected from 0, 1, 2, and 3; and e is an integer between 1 and 10, i.e., e is selected from 1, 2, 3, 4, 5, 6, 7, 8, 9, and 10; preferably, e is an integer between 1 and 6, i.e., e is selected from 1, 2, 3, 4, 5, and 6; and further preferably, e is an integer between 1 and 3, i.e., e is selected from 1, 2, and 3;

Y is a terminal active group selected from the group consisting of —H, —NH₂, —COCH═CH₂, —COC(CH₃)═CH₂,

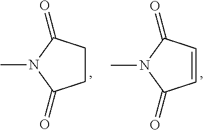

### —SH,

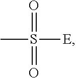

—CHO, —C≡CH, —PO₃H, —N₃, —CN, —CH═CH₂,

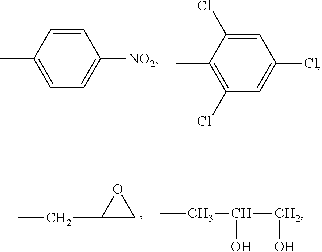

### —CH═CH—COOH, —N═C═O,

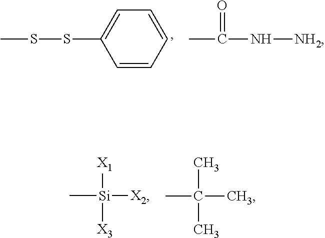

C1-6 alkyl and C1-6 alkoxy; preferably, Y is selected from the group consisting of —H, —NH₂, —COCH═CH₂, —COC(CH₃)═CH₂,

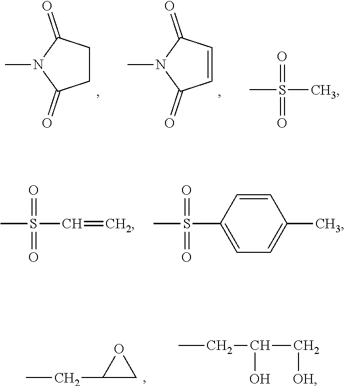

### —CH═CH—COOH, —N═C═O,

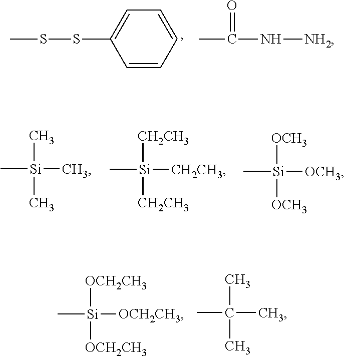

methyl, ethyl, propyl, isopropyl, methoxy, ethoxy, and propoxy; further preferably, Y is selected from the group consisting of —H, —NH₂, —COCH═CH₂,

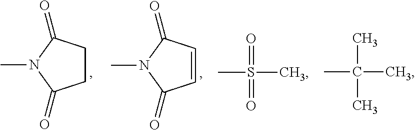

methyl, ethyl, methoxy, and ethoxy; and most preferably, Y is selected from the group consisting of: —H, —NH₂, —COCH═CH₂,

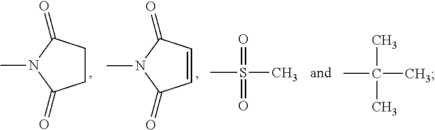

E is a C1-10 hydrocarbyl or a fluorine atom-containing C1-10 hydrocarbyl; preferably, E is selected from the group consisting of methyl, ethyl, propyl, isopropyl, butyl, tert-butyl, pentyl, hexyl, heptyl, octyl, nonyl, decyl, vinyl, phenyl, benzyl, p-methylphenyl, trifluoromethyl, 2,2,2-trifluoroethyl, and 4-(trifluoromethoxy)phenyl; further preferably, E is selected from the group consisting of methyl, ethyl, propyl, butyl, vinyl, phenyl, benzyl, p-methylphenyl, and trifluoromethyl; still further preferably, E is selected from the group consisting of methyl, vinyl, and p-methylphenyl; and most preferably, E is methyl;

X₁, X₂ and X₃ are the same or not the same C1-10 hydrocarbyl or C1-6 alkoxy; preferably, X₁, X₂ and X₃ are independently selected from the group consisting of methyl, ethyl, propyl, isopropyl, butyl, tert-butyl, pentyl, hexyl, heptyl, octyl, nonyl, decyl, phenyl, benzyl, p-methylphenyl, methoxy, ethoxy, and propoxy; further preferably, X₁, X₂ and X₃ are independently selected from the group consisting of methyl, ethyl, propyl, isopropyl, phenyl, benzyl, methoxy, and ethoxy; and most preferably, X₁, X₂ and X₃ are independently selected from the group consisting of methyl, ethyl, methoxy, and ethoxy; and

PEG is the same or not the same —(OCH₂CH₂)ₘ—, and the average value of m is an integer between 3 and 250; preferably, the average value of m is an integer between 10 and 200; further preferably, the average value of m is an integer between 20 and 150; still further preferably, the average value of m is an integer between 20 and 100; and most preferably an integer between 20 and 80.

In a specific embodiment of the present invention, the derivative of the multi-armed polyethylene glycol has structures of III-1 to III-11:

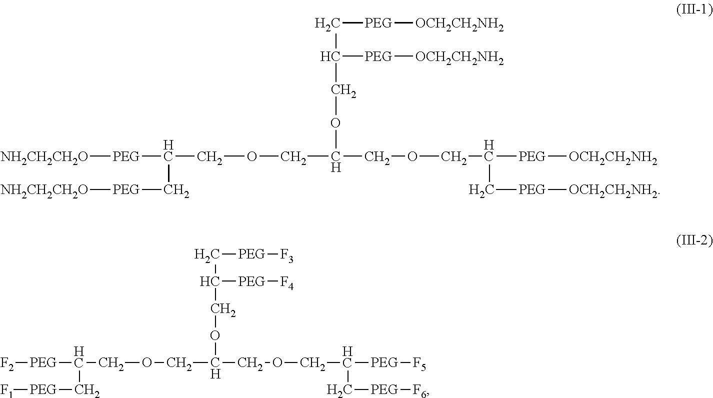

in formula III-2, F₁, F₂, F₃, F₄, F₅ and F₆ are all

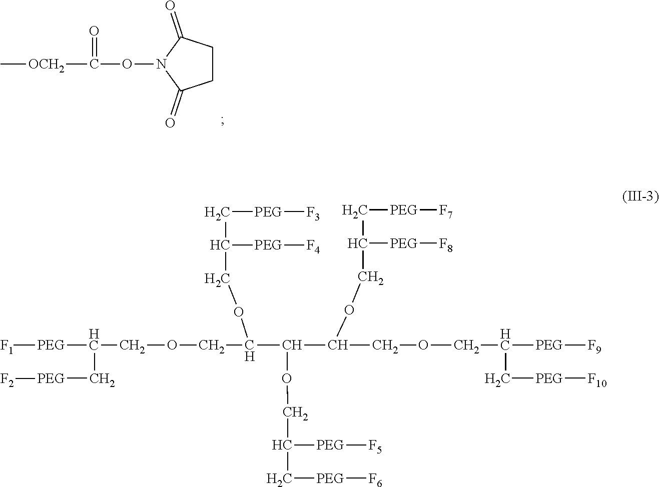

in formula III-3, F₁, F₂, F₃, F₄, F₅, F₆, F₇, F₈, F₉ and F₁₀ are all

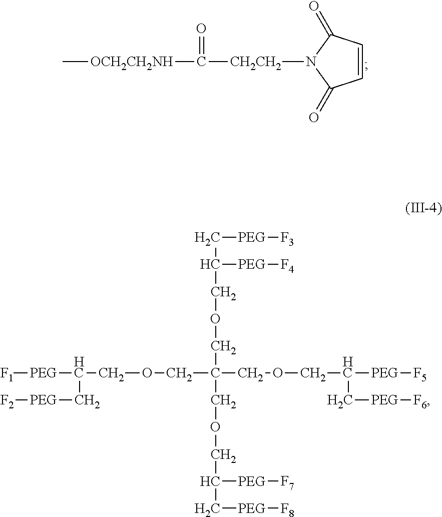

in formula III-4, F₁, F₂, F₃, F₄, F₅, F₆, F₇ and F₈ are all

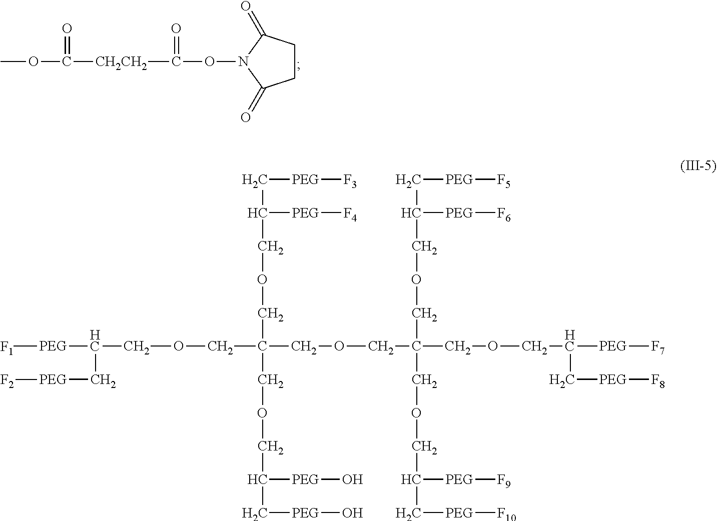

in formula III-5, F₁, F₂, F₃, F₄, F₅, F₆, F₇, F₈, F₉, F₁₀, F₁₁ and F₁₂ are all

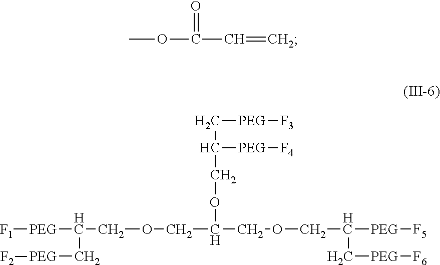

in formula III-6, F₁ is —OCH₂COOH, and F₂, F₃, F₄, F₅ and F₆ are all —OH;

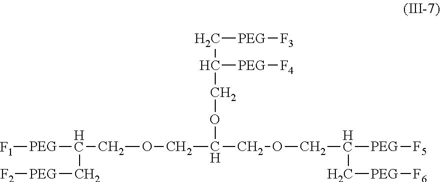

in formula III-7, F₁ is —OCH₂COOH, and F₂, F₃, F₄, F₅ and F₆ are all —OCH₂CH₂—NH₂;

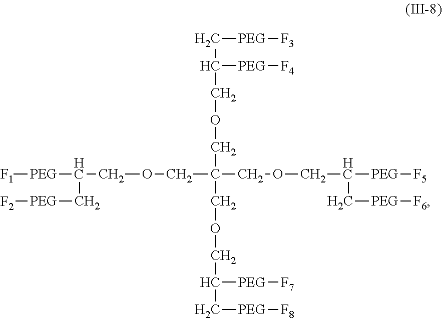

in formula III-8, F₁ is —OCH₂COOH, and F₂, F₃, F₄, F₅, F₆, F₇ and F₈ are all —OH;

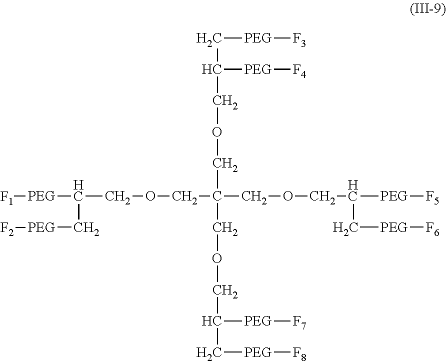

in formula III-9, F₁ and F₂ are —OCH₂COOH, and F₃, F₄, F₅, F₆, F₇ and F₈ are all —OH;

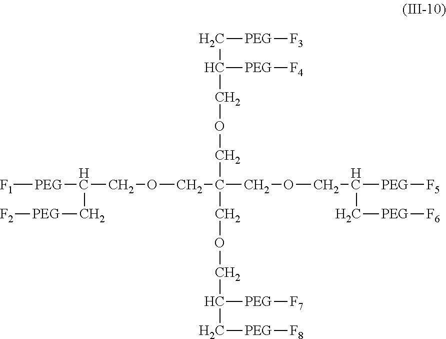

in formula III-10, F₁ is —OCH₂COOH, and F₂, F₃, F₄, F₅, F₆, F₇ and F₈ are all —OCH₂CH₂—NH₂; and

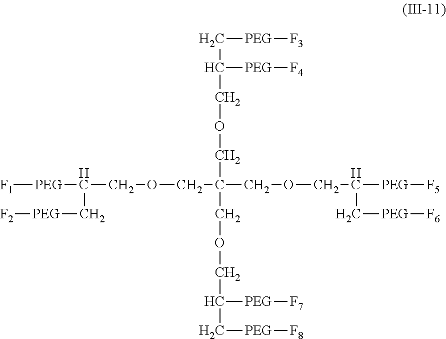

in formula III-11, F₁ is:

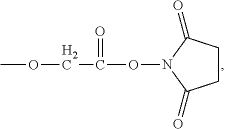

and F₂, F₃, F₄, F₅, F₆, F₇ and F₈ are all:

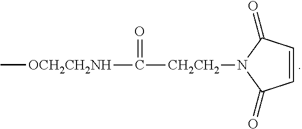

Another object of the present invention is to provide a conjugate of the above-mentioned multi-armed polyethylene glycol active derivative and a drug molecule.

The multi-armed polyethylene glycol reactive derivative forms the conjugate with a drug molecule through its terminal group F.

The drug molecule is selected from the group consisting of amino acids, polypeptides, proteins, nucleosides, saccharides, organic acids, flavonoids, quinines, terpenes, phenylpropyl phenols, steroids and their glycosides, alkaloids, and combinations thereof.

Preferably, the drug molecule is selected from the group consisting of chlorambucil, cis-platinum, 5-fluorouracil, paclitaxel, doxorubicin, methotrexate, interferon, interleukin, tumor necrosis factor, growth factor, colony stimulating factor, hemopoietin, superoxide dismutase, irinotecan, and docetaxel.

More preferably, the drug molecule is irinotecan, or docetaxel.

Most preferably, the drug molecule is irinotecan.

Preferably, the conjugate of the present invention is a conjugate formed by eight-armed polyethylene glycol acetic acid and irinotecan or docetaxel.

Another object of the present invention is to provide a pharmaceutical composition comprising the above-mentioned conjugate formed by the multi-armed polyethylene glycol active derivative and drug molecule, and a pharmaceutically acceptable carrier or excipient.

The pharmaceutical composition is a dosage form of a tablet, a capsule, a pill, a granule, a powder, a suppository, an injection, a solution, a suspension, a plaster, a patch, a lotion, a drop, a liniment, a spray, and the like.

Another object of the present invention is to provide a gel formed by the above-mentioned multi-armed polyethylene glycol active derivative.

The present invention further provides use of the above-mentioned multi-armed polyethylene glycol, active derivative of the multi-armed polyethylene glycol, drug conjugate thereof and gel material in preparing a medicament.

The multi-armed polyethylene glycol prepared by the present invention has a low polydispersity and a relatively high molecular weight, that is, has the characteristics of narrow molecular weight distribution and high purity, wherein the polydispersity and molecular weight are determined by GPC and MALDI, respectively, and the low polydispersity means a polydispersity of less than 1.1. The multi-armed polyethylene glycol and the active derivative thereof provided by the present invention can be used for the modification of a drug, which can improve the solubility, stability and immunogenicity of the drug, improve the absorption of the drug in the body, prolong the half-life and improve the bioavailability of the drug, enhance the curative effect, and reduce the side effects. The gel formed by the multi-armed polyethylene glycol active derivative provided by the present invention can be used for preparing a sustained and controlled release drug, which can prolong the time of the drug action, reduce the number of administrations, and improve patient compliance.

### DETAILED DESCRIPTION OF THE INVENTION

The technical solutions in the embodiments of the present invention will be clearly and completely described below. It is obvious that the described embodiments are only a part of the embodiments of the present invention, and not all of the embodiments. All other embodiments obtained by those skilled in the art based on the embodiments of the present invention without creative efforts are within the scope of the present invention.

As used herein, unless otherwise specified, “polyol” is an alcohol compound having three or more hydroxyl groups in the molecule, such as glycerol, pentaerythritol, polypentaerythritol, trimethylolethane, xylitol (1,2,3,4,5-pentahydroxypentane), sorbitol (1,2,3,4,5,6-hexahydroxyhexane), etc. and derivatives thereof, and “polyol group” is a radical formed by the above-mentioned “polyol” after losing a hydroxyl hydrogen.

As used herein, “multi-armed polyethylene glycol”, also referred to as “multi-armed PEG”, refers to a branched polyethylene glycol in which the branches (“arms”) are terminated with hydroxyl groups.

As used herein, “multi-armed polyethylene glycol” is synonymous with “star polyethylene glycol”, and is a multi-armed polyethylene glycol having a central branching point, which may be a single atom or a chemical group from which a linear arm is emitted.

The multi-armed polyethylene glycol according to the present invention is a multi-armed polyethylene glycol formed by polymerizing ethylene oxide with a polyol glyceryl ether as an initiator. The present invention also relates to improvements in the synthesis process of polyol glyceryl ethers.

For polyethylene glycol, it is generally expressed by molecular weight. Due to the potential heterogeneity of the starting PEG compound, which is generally defined by its average molecular weight rather than the repeating unit, it is preferred to characterize the degree of polymerization of the polyethylene glycol by molecular weight instead of using the integer m to represent the repeating unit in the PEG polymer.

As used herein, “hydrocarbyl” refers to a functional group containing only two kinds of atoms, carbon and hydrogen, and may be divided into an aromatic hydrocarbyl and an aliphatic hydrocarbyl, the former is, for example, phenyl, benzyl, etc., and the latter can be divided into alkyl, alkenyl, alkynyl such as methyl, ethyl, vinyl, ethynyl and the like. The C1-10 hydrocarbyl is a hydrocarbyl having 1 to 10 carbon atoms. The hydrocarbyl may be optionally substituted by one or more substituents, for example, fluorine may optionally replace the hydrogen in a hydrocarbon group.

As used herein, “alkyl” refers to a linear or branched hydrocarbon chain radical containing no unsaturated bond, and which is linked to the rest of the molecule by a single bond. C1-6 alkyl refers to an alkyl having 1 to 6 carbon atoms, such as methyl, ethyl, (n-)propyl, isopropyl, n-butyl, isobutyl, sec-butyl, tert-butyl, n-pentyl, n-hexyl, etc. The alkyl radical may be optionally substituted by one or more substituents, for example, it corresponds to an alkoxy if substituted by oxygen.

Active Groups:

For the use of the multi-armed polyethylene glycol active derivative of the present invention, the difference in the terminal functional groups F determines that the derivatives have different uses. The introduction of these functional groups will determine the field and structure to which the reactive derivative is suitable for application. The most commonly used functional group is N-hydroxysuccinimide ester (NHS). The active derivative with a NHS ester structure can be attached to a group having an amine group.

Likewise, one skilled in the art will be able to obtain a multi-armed polyethylene glycol active derivative having an amino functional group in accordance with the description of the present specification.

Likewise, one skilled in the art will be able to obtain a multi-armed polyethylene glycol active derivative having a carboxyl functional group.

Likewise, one skilled in the art will be able to obtain a multi-armed polyethylene glycol active derivative having a maleimide functional group (MAL). The active derivative having a MAL structure can be attached to a group having a sulfhydryl.

Likewise, the present invention also provides a multi-armed heterofunctional polyethylene glycol polymer that broadens the channel for the application of polyethylene glycol.

As used herein, “gel” refers to a water swellable polymeric matrix composed of a three-dimensional network of macromolecules joined together by covalent bonds or non-covalent crosslink bonds that can absorb a significant amount of water to form an elastomeric gel.

Many pharmaceutical ingredients contain active functional groups such as amino, carboxyl, sulfhydryl, etc., which usually bind to monosaccharides, polysaccharides, nucleosides, polynucleoside, phosphoryl groups, etc. in organisms to form active pharmaceutical structures in organisms.

After the functional group is modified, the polyethylene glycol active derivative can also react with a functional group such as carboxyl, sulfydryl, etc., in a drug to form a linker, to replace the biological organic molecules for drug delivery, thereby effectively overcoming the shortcomings of short half-life and short duration of efficacy in organisms.

The multi-armed polyethylene glycol active derivative of the present invention can bind to a drug molecule using an appropriate terminal functional group (F) which allows free amino, carboxyl, hydroxyl, sulfydryl or other groups in a protein, polypeptide or other natural drug to bind to the PEG derivative. For a small molecule drug, each multi-armed polyethylene glycol molecule can bind to a plurality of the drug molecules. Such PEG derivatives have a high drug loading rate to ensure proper drug concentration and enhance sustained release function, and to improve the physiological role of drug molecules in vivo.

The above various application fields only provide a possible reference model for the pharmaceutical application of the PEG derivative, and the specific use and selection need to be confirmed according to pharmacology, toxicology and clinical experiments.

In the conjugate of the present invention, the drug molecule portion is preferably an amino acid, a polypeptide, a protein, a nucleoside, a saccharide, an organic acid, a flavonoid, a quinine, a terpene, a phenylpropyl phenol, a steroid and a glycoside thereof, an alkaloid or the like. The protein drug molecule portion is further preferably an interferon drug, an EPO drug, an auxin drug, an antibody drug, or the like.

The conjugate of the present invention can be administered in the form of a pure compound or a suitable pharmaceutical composition, using any acceptable means of administration or reagents for a similar purpose. Thus, the mode of administration may be selected by oral, intranasal, rectal, transdermal or injection, in the form of solid, semi-solid, lyophilized powder or liquid medicaments, for example, tablets, suppositories, pills, soft and hard gelatin capsules, powders, solutions, suspensions or aerosols, etc., preferably a unit dosage form suitable for simple administration with precise doses. The composition may comprise a conventional pharmaceutical carrier or excipient and one or more conjugates of the present invention as an active ingredient, in addition to other agents, carriers, adjuvants and the like.

Generally, the pharmaceutical composition may comprise from 1 to about 99% by weight of the conjugate of the present invention, and from 99 to 1% by weight of a suitable pharmaceutical excipient, depending on the mode of administration desired. Preferably, the composition comprises from about 5 to 75% by weight of the conjugate of the present invention, and the balance of a suitable pharmaceutical excipient.

The preferred route of administration is by injection, using a conventional daily dosage regimen which can be adjusted to the severity of the disease. The conjugate or a pharmaceutically acceptable salt thereof of the present invention may also be formulated as an injectable preparation, for example, by dissolving from about 0.5 to about 50% of the active ingredient in a pharmaceutical adjuvant which may be administered in liquid form, examples being water, saline, glucose hydrate, glycerol, ethanol or the like, to form a solution or suspension.

If desired, the pharmaceutical composition of the present invention may further comprise a small amount of auxiliary substances such as wetting or emulsifying agents, pH buffers, antioxidants and the like, e.g., citric acid, sorbitanmonolaurate, triethanolamineoleate, butylatedhydroxytoluene and the like.

### EXAMPLE

The polyol glyceryl ether, the multi-armed polyethylene glycol and the active derivative thereof, the conjugate of the active derivative and drug molecule, and the preparation method thereof of the present invention are described below in connection with the examples, which are not intended to limit the present invention, and the scope of the present invention is defined by the claims.

Unless otherwise stated, the reagents used in the following examples were purchased from Sinopharm Chemical Reagent Beijing Co., Ltd. or other similar common chemical sales companies.

### Example 1: Synthesis of Glycerol Triglyceryl Ether

Synthesis of glycerol triglyceryl ether having the following structure:

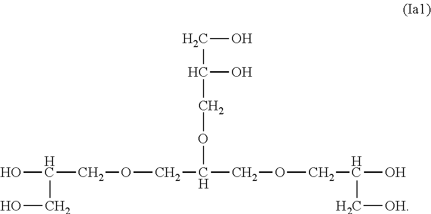 [SMILES: OCC(O)COCC(COCC(O)CO)OCC(O)CO]

To a three-necked flask, glycerol (0.1 mol), dimethyl sulfoxide (100 mL) and potassium hydroxide (0.6 mol) were added, and the mixture was stirred in a water bath. Then epoxy chloropropane (0.9 mol) was added dropwise to the reaction system. The reaction temperature was controlled to not exceed 35° C. The reaction was carried out at room temperature overnight. After the completion of the reaction, the reaction mixture was filtered. The filtrate was washed with dichloromethane. Then the filtrate was collected, rotary evaporated to remove dichloromethane, and finally washed with brine, extracted with ethyl acetate, and rotary evaporated to give a crude product. The crude product is subjected to molecular distillation to obtain pure glycerol glycidyl ether.

The obtained glycerol glycidyl ether (1 g) was dissolved in 10 mL of purified water, and then potassium hydroxide was added thereto to adjust the pH of the reaction liquid to 9-10. The reaction was carried out at 80° C. for 5 hours. After the completion of the reaction, the aqueous phase was rotary evaporated to dryness, and then acetonitrile was added thereto to dissolve the product, which was filtered and rotary evaporated to obtain pure glycerol triglyceryl ether.

¹H-NMR (DMSO-d₆): 3.33-3.48 (m, 16H), 3.47-3.48 (m, 1H), 3.52-3.58 (m, 3H), 4.43 (t, 3H), 4.54 (d, 3H);

ESI (337.2, M+Na);

HPLC detection: the purity of the product was 99.3%.

### Example 2: Synthesis of Butantetraol Tetraglyceryl Ether

Synthesis of butantetraol tetraglyceryl ether having the following structure:

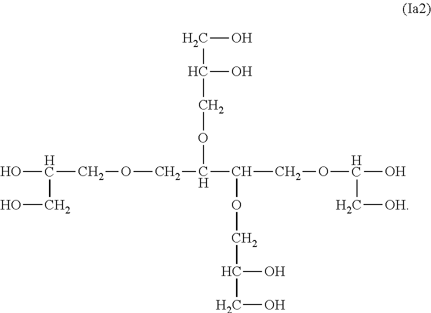 [SMILES: OCC(O)COCC(OCC(O)CO)C(COC(O)CO)OCC(O)CO]

To a three-necked flask, butantetraol (0.1 mol), dimethyl sulfoxide (100 mL) and potassium hydroxide (0.8 mol) were added, and the mixture was stirred in a water bath. Then epoxy chloropropane (1.2 mol) was added dropwise to the reaction system. The reaction temperature was controlled to not exceed 35° C. The reaction was carried out at room temperature overnight. After the completion of the reaction, the reaction mixture was filtered. The filtrate was washed with dichloromethane. Then the filtrate was collected, rotary evaporated to remove dichloromethane, and finally washed with brine, extracted with ethyl acetate, and rotary evaporated to give a crude product. The crude product is subjected to molecular distillation to obtain pure butantetraol glycidyl ether.

The obtained butantetraol glycidyl ether (1 g) was dissolved in 10 mL of purified water, and then potassium hydroxide was added thereto to adjust the pH of the reaction liquid to 9-10. The reaction was carried out at 80° C. for 5 hours. After the completion of the reaction, the aqueous phase was rotary evaporated to dryness, and then acetonitrile was added thereto to dissolve the product, which was filtered and rotary evaporated to obtain pure butantetraol tetraglyceryl ether.

¹H-NMR (DMSO-d₆): 3.33-3.40 (m, 20H), 3.42-3.45 (m, 2H), 3.53-3.57 (m, 4H), 4.41 (t, 4H), 4.52 (d, 4H);

ESI (441.3, M+Na);

HPLC detection: the purity of the product was 99.5%.

### Example 3: Synthesis of Pentitol Pentaglyceryl Ether

Synthesis of pentitol pentaglyceryl ether having the following structure:

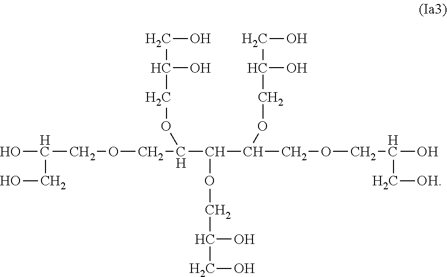 [SMILES: OCC(O)COCC(OCC(O)CO)C(OCC(O)CO)C(COCC(O)CO)OCC(O)CO]

To a three-necked flask, pentitol (0.1 mol), dimethyl sulfoxide (100 mL) and potassium hydroxide (1.0 mol) were added. The mixture was stirred in a water bath.

Then epoxy chloropropane (1.5 mol) was added dropwise to the reaction system. The reaction temperature was controlled to not exceed 35° C. The reaction was carried out at room temperature overnight. After the completion of the reaction, the reaction mixture was filtered. The filtrate was washed with dichloromethane. Then the filtrate was collected, rotary evaporated to remove dichloromethane, and finally washed with brine, extracted with ethyl acetate, and rotary evaporated to give a crude product. The crude product is subjected to molecular distillation to obtain pure pentitol glycidyl ether.

The obtained pentitol glycidyl ether (1 g) was dissolved in 10 mL of purified water, and then potassium hydroxide was added thereto to adjust the pH of the reaction liquid to 9-10. The reaction was carried out at 80° C. for 5 hours. After the completion of the reaction, the aqueous phase was rotary evaporated to dryness, and then acetonitrile was added thereto to dissolve the product, which was filtered and rotary evaporated to obtain pure pentitol pentaglyceryl ether.

¹H-NMR (DMSO-d₆): 3.33-3.40 (m, 24H), 3.43-3.46 (m, 3H), 3.54-3.56 (m, 5H), 4.43 (t, 5H), 4.54 (d, 5H);

ESI (541.4, M+Na);

HPLC detection: the purity of the product was 99.4%.

### Example 4: Synthesis of Hexanehexol Hexaglyceryl Ether

Synthesis of hexanehexol hexaglyceryl ether having the following structure:

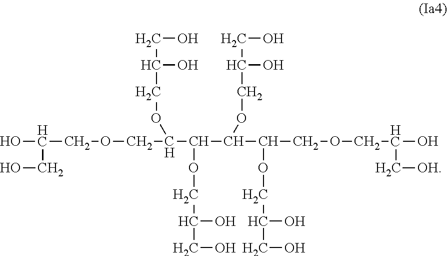 [SMILES: OCC(O)COCC(OCC(O)CO)C(OCC(O)CO)C(OCC(O)CO)C(COCC(O)CO)OCC(O)CO]

To a three-necked flask, hexanehexol (0.1 mol), dimethyl sulfoxide (100 mL) and potassium hydroxide (1.2 mol) were added, and the mixture was stirred in a water bath. Then epoxy chloropropane (1.8 mol) was added dropwise to the reaction system. The reaction temperature was controlled to not exceed 35° C. The reaction was carried out at room temperature overnight. After the completion of the reaction, the reaction mixture was filtered. The filtrate was washed with dichloromethane. Then the filtrate was collected, rotary evaporated to remove dichloromethane, and finally washed with brine, extracted with ethyl acetate, and rotary evaporated to give a crude product. The crude product is subjected to molecular distillation to obtain pure hexanehexol glycidyl ether.

The obtained hexanehexol glycidyl ether (1 g) was dissolved in 10 mL of purified water, and then potassium hydroxide was added thereto to adjust the pH of the reaction liquid to 9-10. The reaction was carried out at 80° C. for 5 hours. After the completion of the reaction, the aqueous phase was rotary evaporated to dryness, and then acetonitrile was added thereto to dissolve the product, which was filtered and rotary evaporated to obtain pure hexanehexol hexaglyceryl ether.

¹H-NMR (DMSO-d₆): 3.32-3.40 (m, 28H), 3.43-3.46 (m, 4H), 3.53-3.56 (m, 6H), 4.44 (t, 6H), 4.53 (d, 6H);

ESI (649.5, M+Na);

HPLC detection: the purity of the product was 99.6%.

### Example 5: Synthesis of Pentaerythritol Tetraglyceryl Ether

Synthesis of pentaerythritol tetraglyceryl ether having the following structure:

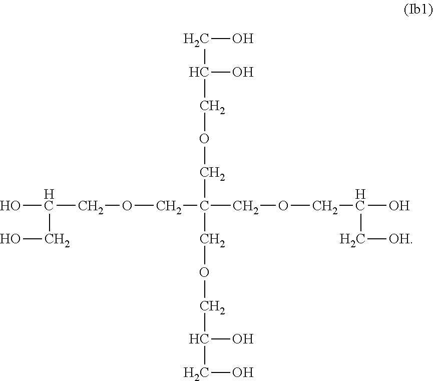 [SMILES: OCC(O)COCC(COCC(O)CO)(COCC(O)CO)COCC(O)CO]

To a three-necked flask, pentaerythritol (0.1 mol), dimethyl sulfoxide (100 mL) and potassium hydroxide (0.8 mol) were added, and the mixture was stirred in a water bath. Then epoxy chloropropane (1.2 mol) was added dropwise to the reaction system. The reaction temperature was controlled to not exceed 35° C. The reaction was carried out at room temperature overnight. After the completion of the reaction, the reaction mixture was filtered. The filtrate was washed with dichloromethane. Then the filtrate was collected, rotary evaporated to remove dichloromethane, and finally washed with brine, extracted with ethyl acetate, and rotary evaporated to give a crude product. The crude product is subjected to molecular distillation to obtain pure pentaerythritol glycidyl ether.

The obtained pentaerythritol glycidyl ether (1 g) was dissolved in 10 mL of purified water, and then potassium hydroxide was added thereto to adjust the pH of the reaction liquid to 9-10. The reaction was carried out at 80° C. for 5 hours. After the completion of the reaction, the aqueous phase was rotary evaporated to dryness, and then acetonitrile was added thereto to dissolve the product, which was filtered and rotary evaporated to obtain pure pentaerythritol tetraglyceryl ether.

¹H-NMR (DMSO-d₆): 3.22-3.40 (m, 24H), 3.52-3.59 (m, 4H), 4.45 (t, 4H), 4.55 (d, 4H);

ESI (455.3, M+Na);

HPLC detection: the purity of the product was 99.4%.

### Example 6: Synthesis of Dipentaerythritol Glyceryl Ether

Synthesis of dipentaerythritol glyceryl ether having the following structure:

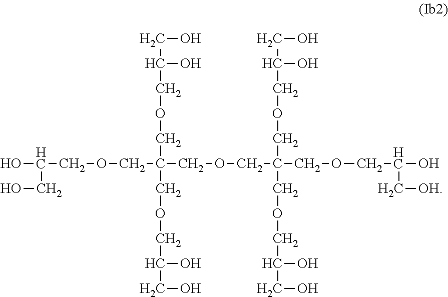 [SMILES: OCC(O)COCC(COCC(O)CO)(COCC(O)CO)COCC(COCC(O)CO)(COCC(O)CO)COCC(O)CO]

To a three-necked flask, dipentaerythritol (0.1 mol), dimethyl sulfoxide (100 mL) and potassium hydroxide (1.2 mol) were added, and the mixture was stirred in a water bath. Then epoxy chloropropane (1.8 mol) was added dropwise to the reaction system. The reaction temperature was controlled to not exceed 35° C. The reaction was carried out at room temperature overnight. After the completion of the reaction, the reaction mixture was filtered. The filtrate was washed with dichloromethane. Then the filtrate was collected, rotary evaporated to remove dichloromethane, and finally washed with brine, extracted with ethyl acetate, and rotary evaporated to give a crude product. The crude product is subjected to molecular distillation to obtain pure dipentaerythritol glycidyl ether.

The obtained dipentaerythritol glycidyl ether (1 g) was dissolved in 10 mL of purified water, and then potassium hydroxide was added thereto to adjust the pH of the reaction liquid to 9-10. The reaction was carried out at 80° C. for 5 hours. After the completion of the reaction, the aqueous phase was rotary evaporated to dryness, and then acetonitrile was added thereto to dissolve the product, which was filtered and rotary evaporated to obtain pure dipentaerythritol glyceryl ether.

¹H-NMR (DMSO-d₆): 3.25-3.42 (m, 40H), 3.52-3.57 (m, 6H), 4.47 (t, 6H), 4.56 (d, 6H);

ESI (721.5, M+Na);

HPLC detection: the purity of the product was 99.2%.

### Example 7: Synthesis of Tripentaerythritol Glyceryl Ether

Synthesis of tripentaerythritol glyceryl ether having the following structure:

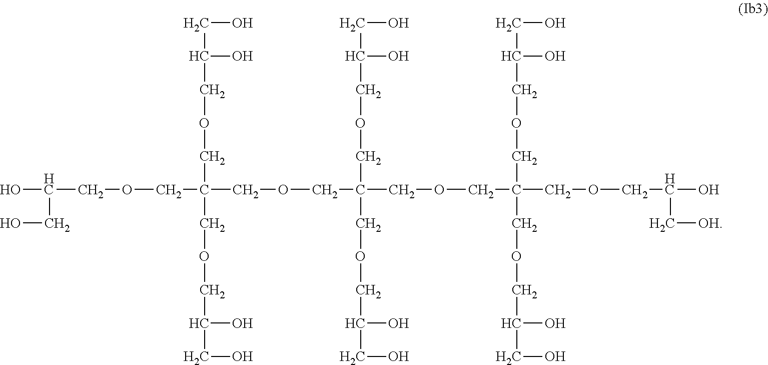 [SMILES: OCC(O)COCC(COCC(O)CO)(COCC(O)CO)COCC(COCC(O)CO)(COCC(O)CO)COCC(COCC(O)CO)(COCC(O)CO)COCC(O)CO]

To a three-necked flask, tripentaerythritol (0.1 mol), dimethyl sulfoxide (100 mL) and potassium hydroxide (1.6 mol) were added, and the mixture was stirred in a water bath. Then epoxy chloropropane (2.4 mol) was added dropwise to the reaction system. The reaction temperature was controlled to not exceed 35° C. The reaction was carried out at room temperature overnight. After the completion of the reaction, the reaction mixture was filtered. The filtrate was washed with dichloromethane. Then the filtrate was collected, rotary evaporated to remove dichloromethane, and finally washed with brine, extracted with ethyl acetate, and rotary evaporated to give a crude product. The crude product is subjected to molecular distillation to obtain pure tripentaerythritol glycidyl ether.

The obtained tripentaerythritol glycidyl ether (1 g) was dissolved in 10 mL of purified water, and then potassium hydroxide was added thereto to adjust the pH of the reaction liquid to 9-10. The reaction was carried out at 80° C. for 5 hours. After the completion of the reaction, the aqueous phase was rotary evaporated to dryness, and then acetonitrile was added thereto to dissolve the product, which was filtered and rotary evaporated to obtain pure tripentaerythritol glyceryl ether.

¹H-NMR (DMSO-d₆): 3.22-3.40 (m, 56H), 3.50-3.54 (m, 8H), 4.45 (t, 8H), 4.56 (d, 8H);

ESI (988.1, M+Na);

HPLC detection: the purity of the product was 99.3%.

### Example 8: Synthesis of Six-Armed Polyethylene Glycol with Glycerol Triglyceryl Ether as Core

Synthesis of six-armed polyethylene glycol having the following structure:

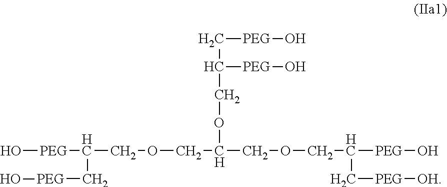

The glycerol triglyceryl ether (31.4 g) prepared in Example 1 and an appropriate amount of a catalyst were placed together in a reaction vessel, heated to 110° C., and vacuumed for 2 hours. 2 kg of ethylene oxide was introduced until the reaction was completed. The product was determined by MALDI to have a number average molecular weight of 20,000.

¹H-NMR (DMSO-d₆): 3.50 (m, hydrogen in —(CH₂CH₂O)—), 4.57 (t, 6H);

GPC detection: the polydispersity was 1.03.

### Example 9: Synthesis of Ten-Armed Polyethylene Glycol with Pentitol Pentaglyceryl Ether as Core

Synthesis of ten-armed polyethylene glycol having the following structure:

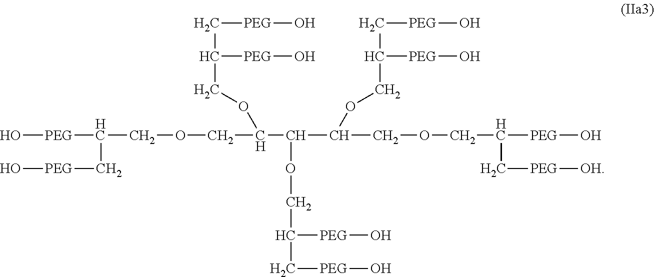

The pentitol pentaglyceryl ether (52.2 g) prepared in Example 3 and an appropriate amount of a catalyst were placed together in a reaction vessel, heated to 110° C., and vacuumed for 2 hours. 2 kg of ethylene oxide was introduced until the reaction was completed. The product was determined by MALDI to have a number average molecular weight of 20,000.

¹H-NMR (DMSO-d₆): 3.50 (m, hydrogen in —(CH₂CH₂O)—), 4.53 (t, 10H);

GPC detection: the polydispersity was 1.03.

### Example 10: Synthesis of Twelve-Armed Polyethylene Glycol with Hexanehexol Hexaglyceryl Ether as Core

Synthesis of twelve-armed polyethylene glycol having the following structure:

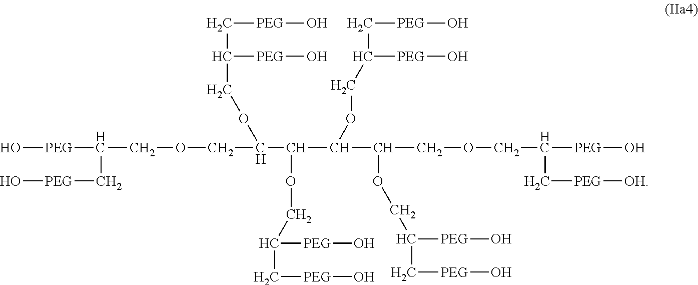

The hexanehexol hexaglyceryl ether (62.6 g) prepared in Example 4 and an appropriate amount of a catalyst were placed together in a reaction vessel, heated to 110° C., and vacuumed for 2 hours. 2 kg of ethylene oxide was introduced until the reaction was completed. The product was determined by MALDI to have a number average molecular weight of 20,000.

¹H-NMR (DMSO-d₆): 3.51 (m, hydrogen in —(CH₂CH₂O)—), 4.57 (t, 12H);

GPC detection: the polydispersity was 1.04.

### Example 11: Synthesis of Eight-Armed Polyethylene Glycol with Pentaerythritol Tetraglyceryl Ether as Core

Synthesis of eight-armed polyethylene glycol having the following structure:

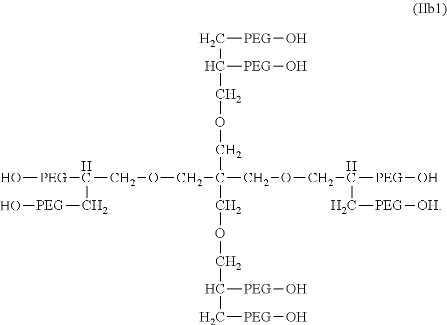

The pentaerythritol tetraglyceryl ether (43.2 g) prepared in Example 5 and an appropriate amount of a catalyst were placed together in a reaction vessel, heated to 110° C., and vacuumed for 2 hours. 1.5 kg of ethylene oxide was introduced until the reaction was completed. The product was determined by MALDI to have a number average molecular weight of 15,000.

¹H-NMR (DMSO-d₆): 3.50 (m, hydrogen in —(CH₂CH₂O)—), 4.56 (t, 8H);

GPC detection: the polydispersity was 1.03.

### Example 12: Synthesis of Twelve-Armed Polyethylene Glycol with Dipentaerythritol Hexaglyceryl Ether as Core

Synthesis of twelve-armed polyethylene glycol having the following structure:

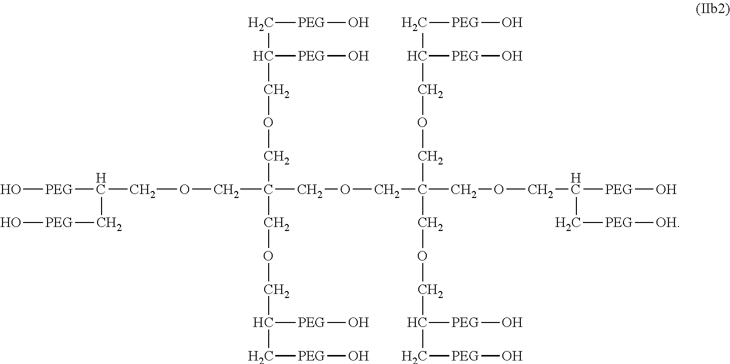

The dipentaerythritol hexaglyceryl ether (69.8 g) prepared in Example 6 and an appropriate amount of a catalyst were placed together in a reaction vessel, heated to 110° C., and vacuumed for 2 hours. 1.95 kg of ethylene oxide was introduced until the reaction was completed. The product was determined by MALDI to have a number average molecular weight of 20,000.

¹H-NMR (DMSO-d₆): 3.50 (m, hydrogen in —(CH₂CH₂O)—), 4.57 (t, 12H);

GPC detection: the polydispersity was 1.04.

### Example 13: Synthesis of Six-Armed Polyethylene Glycol-Amine with Glycerol Glyceryl Ether as Core

Synthesis of six-armed polyethylene glycol-amine having the following structure:

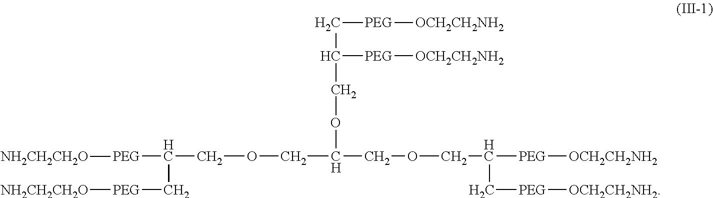

20 g of six-armed polyethylene glycol having a number average molecular weight of 20,000 (prepared in Example 8) was azeotroped with toluene for two hours under a nitrogen atmosphere to remove water, and then cooled to room temperature. 200 mL of dry dichloromethane and 1.2 mL of triethylamine were added thereto. Dry methanesulfonyl chloride was added dropwise under condition of ice bath. The mixture was stirred overnight under a nitrogen atmosphere. 3 mL of absolute ethanol was added thereto to quench the reaction. The solvent was concentrated by rotary evaporation. After recrystallization, the precipitate was collected and dried in vacuum to give a six-armed polyethylene glycol-methylsulfonyl ester having a number average molecular weight of 20,000 in a yield of 95%.

10 g of six-armed polyethylene glycol-methylsulfonyl ester having a number average molecular weight of 20,000 (prepared in the previous step) was dissolved in 100 mL of an aqueous ammonia solution containing 5% ammonium chloride. The solution was allowed to react at room temperature for 72 hours, and then the reaction was terminated. After the completion of the reaction, the reaction mixture was extracted three times with dichloromethane. The organic phases were combined and dried over anhydrous sodium sulfate, rotary evaporated to remove solvent, and then recrystallized. The precipitate was collected and dried in vacuum to give a six-armed polyethylene glycol-amine in a yield of 70%.

¹H-NMR (DMSO-d₆): 2.61 (t, 6×2H), 3.50 (m, hydrogen in —(CH₂CH₂O)—).

### Example 14: Synthesis of Six-Armed Polyethylene Glycol-Acetic Acid-NHS Ester with Glycerol Glyceryl Ether as Core

Synthesis of six-armed polyethylene glycol-acetic acid-NHS ester having the following structure:

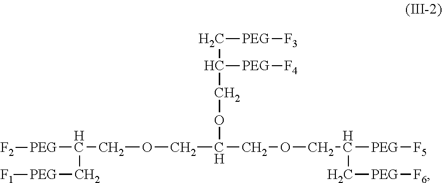

wherein, F₁, F₂, F₃, F₄, F₅, and F₆ are all:

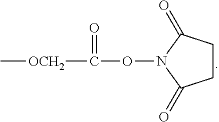

20 g of six-armed polyethylene glycol having a number average molecular weight of 20,000 (prepared in Example 8) was azeotroped with toluene for two hours under a nitrogen atmosphere to remove water, and then cooled to 50° C., followed by addition of 2 g of potassium t-butoxide. The mixture was reacted at 50° C. for 2 hours, and then cooled to room temperature. 2 mL of t-butyl bromoacetate was added thereto. The mixture was reacted overnight at room temperature under the protection of nitrogen. After the completion of the reaction, the mixture was concentrated by rotary evaporation, and then added with 200 mL of isopropanol for precipitation, and filtered. The filter cake was collected, and dried in vacuum to give six-armed polyethylene glycol-tert-butyl acetate.

200 mL of NaOH solution with pH of 12 was prepared. The six-armed polyethylene glycol-tert-butyl acetate obtained in the previous step was hydrolyzed overnight. After the hydrolysis overnight, the reaction solution was adjusted to pH 2 with concentrated hydrochloric acid, dissolved by adding 20 g of sodium chloride and stirring, and extracted three times with dichloromethane. The organic phases were combined, and dried over anhydrous sodium sulfate. The organic phase was concentrated, precipitated with 300 mL of isopropanol, washed and dried in vacuum to give six-armed polyethylene glycol-acetic acid in a yield of 78%.

The six-armed polyethylene glycol-acetic acid obtained in the previous step was dissolved in 150 mL of dichloromethane. 0.8 g of N-hydroxysuccinimide and 1.6 g of dicyclohexylcarbodiimide were added to the solution. The mixture was stirred at room temperature for 5 hours, evaporated to dryness by rotary evaporation, and then precipitated by adding 150 mL of isopropanol, and filtered. The filter cake was collected, and dried to give the product, six-armed polyethylene glycol-acetic acid-NHS ester, in a yield of 92%.

¹H-NMR (DMSO-d₆): 2.81 (s, 6×4H), 3.50 (m, hydrogen in —(CH₂CH₂O)—), 4.58 (s, 6×2H).

### Example 15: Synthesis of Ten-Armed Polyethylene Glycol-Maleimide with Pentitol Pentaglyceryl Ether as Core

Synthesis of ten-armed polyethylene glycol-maleimide having the following structure:

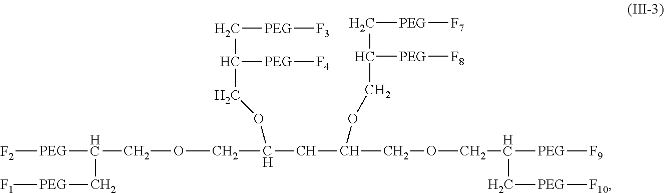

wherein, F₁, F₂, F₃, F₄, F₅, F₆, F₇, F₈, F₉, and F₁₀ are all:

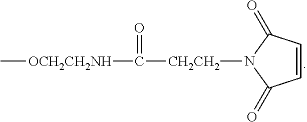

20 g of ten-armed polyethylene glycol having a number average molecular weight of 20,000 (prepared in Example 9) was azeotroped with toluene for two hours under a nitrogen atmosphere to remove water, and then cooled to room temperature. 200 mL of dry dichloromethane and 2.0 mL of triethylamine were added thereto. Dry methanesulfonyl chloride was added dropwise under condition of ice bath. The mixture was stirred overnight under a nitrogen atmosphere. 3 mL of absolute ethanol was added to quench the reaction. The solvent was concentrated by rotary evaporation. After recrystallization, the precipitate was collected and dried in vacuum, to give ten-armed polyethylene glycol-methylsulfonyl ester having a number average molecular weight of 20,000 in a yield of 93%.

The polyethylene glycol-methylsulfonyl ester obtained in the previous step was dissolved in 200 mL of an aqueous ammonia solution containing 5% ammonium chloride. The solution was allowed to react at room temperature for 72 hours, and then the reaction was terminated. After the completion of the reaction, the reaction mixture was extracted three times with dichloromethane. The organic phases were combined and dried over anhydrous sodium sulfate, rotary evaporated to remove solvent, and then recrystallized. The precipitate was collected and dried in vacuum to give ten-armed polyethylene glycol-amine in a yield of 71%.

The ten-armed polyethylene glycol-amine prepared in the previous step was dissolved in acetonitrile. 3.2 g of N-succinimidyl 3-maleimidopropionate was added to the solution. The solution was stirred at room temperature overnight, evaporated to dryness by rotary evaporation, and then added with 300 mL of isopropanol. The precipitate was filtered and dried in vacuum to give the product, ten-armed polyethylene glycol-maleimide, in a yield of 83%.

¹H-NMR (DMSO-d₆): 2.56 (t, 10×2H), 3.50 (m, hydrogen in —(CH₂CH₂O)—), 6.71 (s, 10×2H).

### Example 16: Synthesis of Eight-Armed Polyethylene Glycol-Succinic Acid-NHS Ester with Pentaerythritol Glyceryl Ether as Core

Synthesis of eight-armed polyethylene glycol-succinic acid-NHS ester having the following structure:

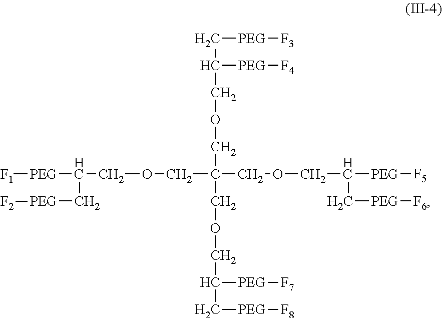

wherein, F₁, F₂, F₃, F₄, F₅, F₆, F₇, and F₈ are all:

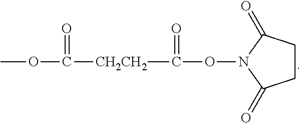

To 15 g of eight-armed polyethylene glycol having a number average molecular weight of 15,000 (prepared in Example 11), 150 mL of toluene was added. 100 mL of toluene was distilled off under the protection of nitrogen. After the solution was cooled to 50° C., 1.0 g of succinic anhydride was added thereto. The mixture was refluxed and reacted for 6 hours, cooled to room temperature, rotary evaporated, precipitated with 150 mL of isopropanol, and filtered. The precipitate was dried to give a crude product.

The crude product obtained in reaction of the previous step was dissolved in 150 mL of dichloromethane. 1.0 g of N-hydroxysuccinimide and 2.2 g of dicyclohexylcarbodiimide were added to the solution. The mixture was stirred at room temperature for 6 hours. After the completion of the reaction, the reaction mixture was rotary evaporated to remove the solvent, and then was precipitated with 150 mL of isopropanol. The filter cake was collected and dried in vacuum to give the product, eight-armed polyethylene glycol-succinic acid-NHS ester, in a yield of 91%.

¹H-NMR (DMSO-d₆): 2.58 (t, 8×2H), 2.81 (s, 8×4H), 2.93 (t, 8×2H), 3.50 (m, hydrogen in —(CH₂CH₂O)—), 4.28 (t, 8×2H).

### Example 17: Synthesis of Twelve-Armed Polyethylene Glycol-Acrylate with Dipentaerythritol Hexaglyceryl Ether as Core

Synthesis of twelve-armed polyethylene glycol-acrylate having the following structure:

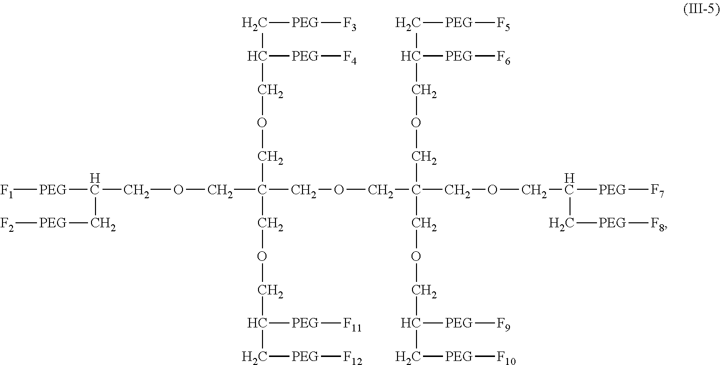

wherein, F₁, F₂, F₃, F₄, F₅, F₆, F₇, F₈, F₉, F₁₀, F₁₁, and F₁₂ are all:

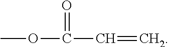

To 20 g of eight-armed polyethylene glycol having a number average molecular weight of 20,000 (prepared in Example 12), 200 mL of toluene was added. 50 mL of toluene was distilled off under the protection of nitrogen. Then the remaining toluene was distilled off under reduced pressure. After adding 200 mL of dichloromethane, the mixture was stirred for 10 minutes in an ice-water bath. Then 1.8 mL of triethylamine was added thereto, and finally 1.2 mL of acryloyl chloride was added dropwise thereto. The mixture was ice-water-bathed for 1 hour, and reacted at room temperature for 5 hours to complete the reaction. After the completion of the reaction, the reaction mixture was rotary evaporated to dryness. The residue was precipitated with 200 mL of isopropanol, and filtered. The filter cake was collected, and dried in vacuum to give the product, twelve-armed polyethylene glycol-acrylate, in a yield of 88%.

¹H-NMR (DMSO-d₆): 3.50 (m, hydrogen in —(CH₂CH₂O)—), 4.21 (t, 12×2H), 5.96 (q, 12×1H), 6.19 (q, 12×1H), 6.34 (q, 12×1H).

### Example 18: Synthesis of Six-Armed Polyethylene Glycol-Hydroxy-Monoacetic Acid with Glycerol Glyceryl Ether as Core

Synthesis of six-armed polyethylene glycol-hydroxy-monoacetic acid having the following structure:

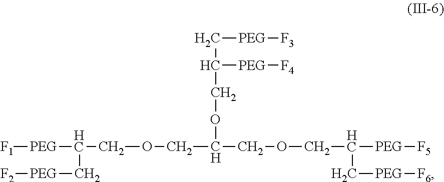

wherein, F₁ is —OCH₂COOH, and F₂, F₃, F₄, F₅, and F₆ are all hydroxy.

20 g of six-armed polyethylene glycol having a molecular weight of 20,000 was dehydrated with 100 mL of toluene. Then the remaining toluene was distilled off. 200 mL of tetrahydrofuran and 0.14 g of potassium t-butoxide were added thereto. The mixture was reacted at room temperature for 2 hours. Then 0.25 g of t-butyl bromoacetate was added dropwise. The mixture was reacted at room temperature overnight, and then filtered. The filtrate was concentrated by rotary evaporation, then added with 100 mL of NaOH solution (1 mol/L), and subjected to alkaline hydrolysis at 80° C. for 2 hours, then adjusted to pH 2-3 with 2N hydrochloric acid, and then added with 10 g of NaCl, and extracted three times with dichloromethane. The organic phases were combined, dried over anhydrous sodium sulfate, filtered, concentrated by rotary evaporation, precipitated with diethyl ether and dried in vacuum. The crude product was separated by a DEAE anion exchange resin column, and different fractions were separately collected to obtain six-armed polyethylene glycol-hydroxy-monoacetic acid fraction. The product structure was determined by ¹H-NMR.

Six-armed polyethylene glycol-hydroxy-monoacetic acid ¹H-NMR (DMSO-d₆): 3.50 (m, hydrogen in —(CH₂CH₂O)—), 4.01 (t, 1×2H).

### Example 19: Synthesis of Six-Armed Polyethylene Glycol-Amine-Monoacetic Acid with Glycerol Glyceryl Ether as Core

Synthesis of six-armed polyethylene glycol-amine-monoacetic acid having the following structure:

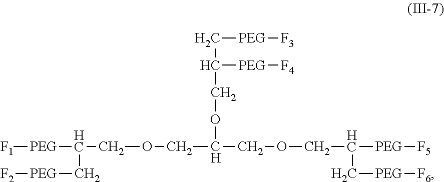

wherein, F₁ is —OCH₂COOH, and F₂, F₃, F₄, F₅, and F₆ are all —OCH₂CH₂—NH₂.

20 g of six-armed polyethylene glycol-hydroxy-monoacetic acid having a number average molecular weight of 20,000 (prepared in Example 18) was dissolved in 200 mL of anhydrous methanol, and ice-water-bathed. 10 mL of concentrated hydrochloric acid was added dropwise. The mixture was reacted for 3 hours at room temperature. After completion of the reaction, the reaction mixture was adjusted to pH 7.0 with a 8% sodium bicarbonate aqueous solution, and extracted three times with dichloromethane. The organic phases were combined, dried over anhydrous sodium sulfate, filtered, and concentrated by rotary evaporation to give a crude product, which was precipitated with diethyl ether to give six-armed polyethylene glycol-hydroxy-monomethylacetate.

To the six-armed polyethylene glycol-hydroxy-monomethylacetate synthesized in the previous step, 100 mL of toluene was added. The mixture was rotary evaporated to remove water, and then evaporated to dryness by rotary evaporation to remove the toluene. The residue was dissolved in 200 mL of dichloromethane. Then 1.0 mL of triethylamine was added thereto. The mixture was stirred for 10 minutes in an ice-water bath. Then 0.69 g of methylsulfonyl chloride was added dropwise thereto. The mixture was ice-water-bathed for 1 hour and then reacted at room temperature overnight. After the completion of the reaction, the reaction mixture was added with 200 mL of distilled water, and extracted twice with dichloromethane. The organic phases were combined, dried over anhydrous sodium sulfate, filtered, and rotary evaporated to give a crude product of six-armed polyethylene glycol-sulfonate-monomethylacetate.

The crude product of six-armed polyethylene glycol-sulfonate-monomethylacetate synthesized in the previous step was dissolved in 45 mL of de-aerated water. The reaction solution was adjusted to pH 12.0 with a 2N sodium hydroxide aqueous solution. The mixture was reacted at room temperature for 2-3 hours. Then, 100 mL of an aqueous ammonia solution in which 5.2 g of ammonium chloride was dissolved was added to the reaction. The mixture was reacted at room temperature for 72 hours. After completion of the reaction, the reaction mixture was added with saturated brine, and extracted three times with dichloromethane. The organic phases were combined and concentrated by rotary evaporation. Then the residue is dissolved in 100 mL of water, adjusted to pH 2-3 with 2N hydrochloric acid, added with sodium chloride, and then extracted three times with dichloromethane. The organic phases were combined, dried over anhydrous sodium sulfate, filtered, concentrated by rotary evaporation, and then recrystallized from diethyl ether to give six-armed polyethylene glycol-amine-monoacetic acid in a yield of 86%.

¹H-NMR (DMSO-d₆): 2.96 (t, 5×2H), 3.50 (m, hydrogen in —(CH₂CH₂O)—), 4.40 (t, 1×2H).

### Example 20: Synthesis of Eight-Armed Polyethylene Glycol-Hydroxy-Monoacetic Acid and Eight-Armed Polyethylene Glycol-Hydroxy-Diacetic Acid

Synthesis of eight-armed polyethylene glycol-hydroxy-monoacetic acid having the following structure:

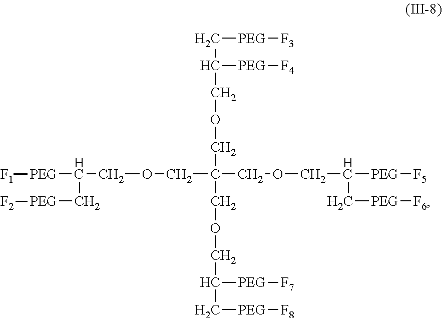

wherein, F₁ is —OCH₂COOH, and F₂, F₃, F₄, F₅, F₆, F₇, and F₈ are all hydroxy;

and eight-armed polyethylene glycol-hydroxy-diacetic acid having the following structure:

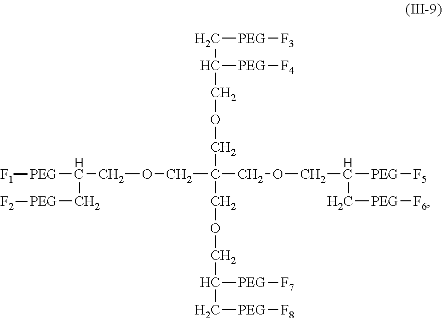

wherein, F₁ and F₂ are —OCH₂COOH, and F₃, F₄, F₅, F₆, F₇, and F₈ are all hydroxy.

200 g of eight-armed polyethylene glycol having a molecular weight of 20,000 was dehydrated with 100 mL of toluene. Then the remaining toluene was distilled off. 750 mL of tetrahydrofuran and 2.24 g of potassium t-butoxide were added. The mixture was reacted at room temperature for 2 hours. Then 3.90 mL of t-butyl bromoacetate was added dropwise thereto. The mixture was reacted at room temperature overnight, and then filtered. The filtrate was concentrated by rotary evaporation, then added with 500 mL of NaOH solution (1 mol/L), and subjected to alkaline hydrolysis at 80° C. for 2 hours, then adjusted to pH 2-3 with 2N hydrochloric acid, and then added with 50 g of NaCl, and extracted three times with dichloromethane. The organic phases were combined, dried over anhydrous sodium sulfate, filtered, concentrated by rotary evaporation, precipitated with diethyl ether and dried in vacuum. The crude product was separated by a DEAE anion exchange resin column, and different fractions were separately collected to obtain eight-armed polyethylene glycol-hydroxy-monoacetic acid and eight-armed polyethylene glycol-hydroxy-diacetic acid fractions, respectively. The product structures were determined by ¹H-NMR.

Eight-armed polyethylene glycol-hydroxy-monoacetic acid ¹H-NMR (DMSO-d₆): 3.50 (m, hydrogen in —(CH₂CH₂O)—), 4.01 (t, 1×2H);

Eight-armed polyethylene glycol-hydroxy-diacetic acid ¹H-NMR (DMSO-d₆): 3.50 (m, hydrogen in —(CH₂CH₂O)—), 4.01 (t, 2×2H).

### Example 21: Synthesis of Eight-Armed Polyethylene Glycol-Amine-Monoacetic Acid

Synthesis of eight-armed polyethylene glycol-amine-monoacetic acid having the following structure:

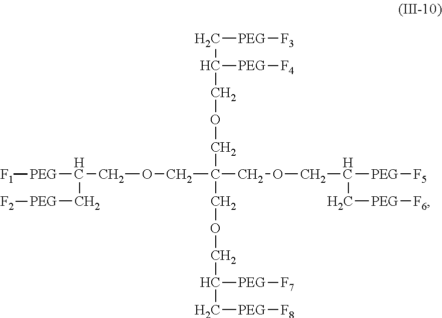

wherein, F₁ is —OCH₂COOH, and F₂, F₃, F₄, F₅, F₆, F₇ and F₈ are all —OCH₂CH₂—NH₂.

200 g of eight-armed polyethylene glycol-hydroxy-monoacetic acid having a number average molecular weight of 20,000 (prepared in Example 20) was dissolved in 750 mL of anhydrous methanol, and ice-water-bathed. 20 mL of concentrated hydrochloric acid was added dropwise thereto. The mixture was reacted for 3 hours at room temperature. After completion of the reaction, the reaction mixture was adjusted to pH 7.0 with a 8% sodium bicarbonate aqueous solution, and extracted three times with dichloromethane. The organic phases were combined, dried over anhydrous sodium sulfate, filtered, and concentrated by rotary evaporation to give a crude product, which was precipitated with diethyl ether to give eight-armed polyethylene glycol-hydroxy-monomethylacetate.

To 100 g of the eight-armed polyethylene glycol-hydroxy-monomethylacetate synthesized in the previous step, 500 mL of toluene was added. The mixture was rotary evaporated to remove water, and evaporated to dryness by rotary evaporation to remove the toluene. The residue was dissolved in 400 mL of dichloromethane, and then 7.4 mL of triethylamine was added thereto. The mixture was stirred for 10 minutes in an ice-water bath. Then 4 mL of methylsulfonyl chloride was added dropwise thereto. The mixture was ice-water-bathed for 1 hour, and then reacted at room temperature overnight. After the completion of the reaction, the reaction mixture was added with 500 mL of distilled water, and extracted twice with dichloromethane. The organic phases were combined, dried over anhydrous sodium sulfate, filtered, and rotary evaporated to give a crude product of eight-armed polyethylene glycol-sulfonate-monomethylacetate.

20 g of the crude product of eight-armed polyethylene glycol-sulfonate-monomethylacetate synthesized in the previous step was dissolved in 45 mL of de-aerated water. The reaction solution was adjusted to pH 12.0 with a 2N sodium hydroxide aqueous solution, and reacted at room temperature for 2-3 hours. Then, 100 mL of an aqueous ammonia solution in which 5.2 g of ammonium chloride was dissolved was added to the reaction. The mixture was reacted at room temperature for 72 hours. After completion of the reaction, the reaction mixture was added with saturated brine, and extracted three times with dichloromethane. The organic phases were combined and concentrated by rotary evaporation. Then the residue is dissolved in 100 mL of water, adjusted to pH 2-3 with 2N hydrochloric acid, added with sodium chloride, and then extracted three times with dichloromethane. The organic phases were combined, dried over anhydrous sodium sulfate, filtered, concentrated by rotary evaporation, and then recrystallized from diethyl ether to give eight-armed polyethylene glycol-amine-monoacetic acid in a yield of 86%.

¹H-NMR (DMSO-d₆): 2.96 (t, 7×2H), 3.50 (m, hydrogen in —(CH₂CH₂O)—), 4.40 (t, 1×2H).

### Example 22: Synthesis of Eight-Armed Polyethylene Glycol-Maleimide-Mono NHS Ester

Synthesis of eight-armed polyethylene glycol-maleimide-mono NHS ester having the following structure:

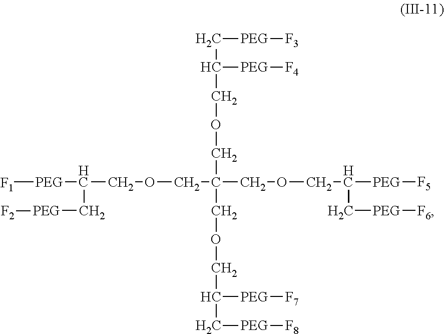

wherein, F₁ is

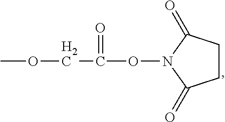

and F₂, F₃, F₄, F₅, F₆, F₇ and F₈ are all

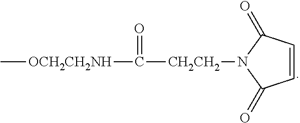

20 g of eight-armed polyethylene glycol-amine-monoacetic acid having a molecular weight of 20,000 was dissolved in 200 mL of dichloromethane. Nitrogen gas was introduced. 1.1 mL of triethylamine was added thereto. The mixture was stirred for 5 minutes. Then 2.4 g of N-succinimidyl 3-maleimidopropionate was added thereto. The mixture was reacted in the dark overnight. After completion of the reaction, the reaction mixture was concentrated to dryness, precipitated with 200 mL of isopropanol in an ice-water bath, filtered, and dried to give eight-armed polyethylene glycol-maleimide-monoacetic acid.

10 g of the crude product of eight-armed polyethylene glycol-hetamaleimide-monoacetic acid obtained in the previous step was dissolved in 100 mL of dichloromethane. Then 0.075 g of N-hydroxysuccinimide was added thereto. After stirring for 10 minutes, the mixture was added with 0.15 g of dicyclohexylcarbodiimide, and reacted at room temperature overnight. After completion of the reaction, the reaction mixture was filtered, concentrated by rotary evaporation, precipitated with 75 mL of isopropanol by hot-melt and ice-water bath, filtered, and dried in vacuum to give eight-armed polyethylene glycol-maleimide-mono NHS ester in a yield of 81%.

¹H-NMR (DMSO-d₆): 2.83 (s, 1×4H), 3.50 (m, hydrogen in —(CH₂CH₂O)—), 4.60 (s, 1×2H), 7.00 (s, 7×2H).

### Example 23: Conjugate of Eight-Armed Polyethylene Glycol-Maleimide-Monoacetic Acid and Irinotecan Derivative

2 g of eight-armed polyethylene glycol-maleimide-monoacetic acid having a number average molecular weight of 20,000 (prepared in Example 22) was dissolved in 20 mL of dichloromethane. Then 0.12 g of irinotecan glycinate (Glycine-Irrinitecan), 50 mg of dimethylaminopyridine and 95 mg of dicyclohexylcarbodiimide were added thereto. The mixture was reacted at room temperature for 6 hours, concentrated by rotary evaporation, then dissolved in 30 mL of dioxane, and filtered. The filtrate was concentrated by rotary evaporation, and then added with 30 mL diethyl ether for precipitation. The precipitate was dried in vacuum to give the product in a yield of 90%.

### Example 24: Synthesis of Stable Gel of Eight-Armed Polyethylene Glycol-Loaded Drug

0.5 g of the conjugate of eight-armed polyethylene glycol maleimide-monoacetic acid and irinotecan derivative having a number average molecular weight of 20,000 (prepared in Example 23) was dissolved in 10 mL of phosphate buffer (pH=7.4). 0.4 g of four-armed polyethylene glycol-SH having a number average molecular weight of 5,000 (available from Beijing Jenkem Technology Co., Ltd., product model: 4ARM-5000-SH) was dissolved in 10 mL of phosphate buffer (pH=7.4). The two were quickly mixed and allowed to stand, and an eight-armed polyethylene glycol gel was formed within 2 minutes. The gel formed was placed in 100 mL of phosphate buffer (pH=7.4), and stored at 37° C. The gel was stable for 360 days without degradation and insolubilization, and the irinotecan in the gel was slowly released.

The above is only the preferred embodiment of the present invention, and is not intended to limit the present invention. Any modifications, equivalent substitutions, etc. made within the spirit and scope of the present invention are intended to be included within the scope of the present invention.

1. A multi-armed polyethylene glycol active derivative having a structure of formula III: 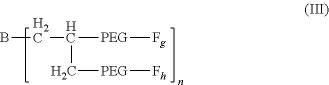
wherein, B is a polyol group, n is an integer between 3 and 22;
F_{g} and Fₕ are the same or not the same —Z—Y type structure;
Z is a linking group selected from the group consisting of —O(CH₂)ᵢ—, —O(CH₂)ᵢNH—, —O(CH₂)ᵢOCOO—, —O(CH₂)ᵢOCONH—, —O(CH₂)ᵢNHCOO—, —O(CH₂)ᵢNHCONH—, —OCO(CH₂)ᵢCOO—, —O(CH₂)ᵢCOO—, —O(CH₂)ᵢCONH— and —O(CH₂)ᵢNHCO(CH₂)ₑ—; i is an integer between 0 and 10, and e is an integer between 1 and 10;
Y is a terminal active group, and
PEG is the same or not the same —(OCH₂CH₂)ₘ—, and the average value of m is an integer between 3 and 250.

2. The multi-armed polyethylene glycol active derivative of claim 1, wherein Y is selected from the group consisting of —H, —NH₂, —COCH═CH₂, —COC(CH₃)═CH₂, 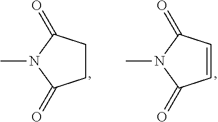
—SH, 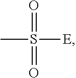
—CHO, —C≡CH, —PO₃H, —N₃, —CN, —CH═CH₂, 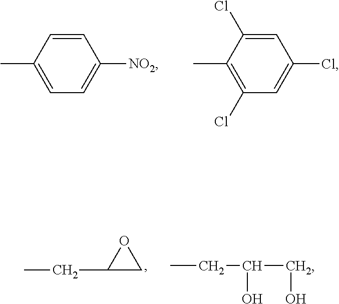
—CH═CH—COOH, —N═C═O, 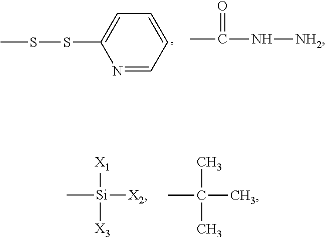
C1-6 alkyl and C1-6 alkoxy;
E is a C1-10 hydrocarbyl or a fluorine atom-containing C1-10 hydrocarbyl; and
X₁, X₂ and X₃ are the same or not the same C1-10 hydrocarbyl or C1-6 alkoxy.

3. The multi-armed polyethylene glycol active derivative of claim 2, wherein E is selected from the group consisting of methyl, ethyl, propyl, butyl, vinyl, phenyl, benzyl, p-methylphenyl, and trifluoromethyl; and
X₁, X₂ and X₃ are independently selected from the group consisting of methyl, ethyl, propyl, isopropyl, phenyl, benzyl, methoxy, and ethoxy.

4. The multi-armed polyethylene glycol active derivative of claim 1, wherein the polyol group B has a structure of formula B₁ or B₂: 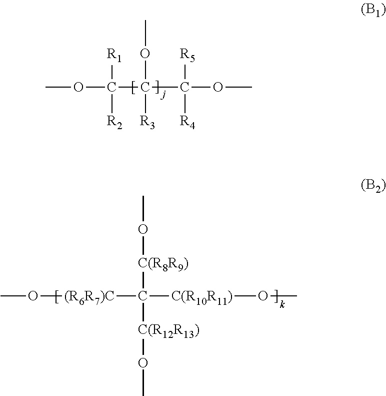
wherein, R₁-R₁₃ are independently selected from the group consisting of —H, C1-10 substituted or unsubstituted alkyl, substituted or unsubstituted aryl, substituted or unsubstituted aralkyl, substituted or unsubstituted aromatic or non-aromatic heterocyclic group; and
j and k are independently selected from integers between 1 and 10.

5. The multi-armed polyethylene glycol active derivative of claim 4, wherein B has a structure of:

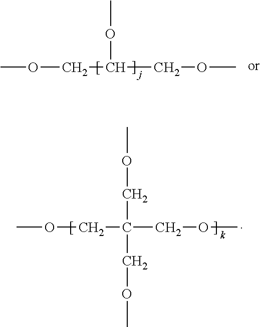

6. The multi-armed polyethylene glycol active derivative of claim 5, wherein j and k are independently selected from integers between 1 and 6.

7. The multi-armed polyethylene glycol active derivative of claim 1, wherein the multi-armed polyethylene glycol active derivative has a number average molecular weight of 1,500 to 80,000.

8. The multi-armed polyethylene glycol active derivative of claim 1, wherein the multi-arm polyethylene glycol active derivative is selected from the following structures: 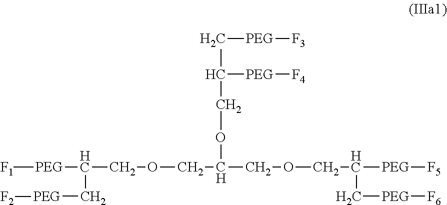
in formula IIIa1, F₁-F₆ are the same or not the same —Z—Y type structures,
Z₁-Z₆ are linking groups, and
Y₁-Y₆ are terminal active groups; 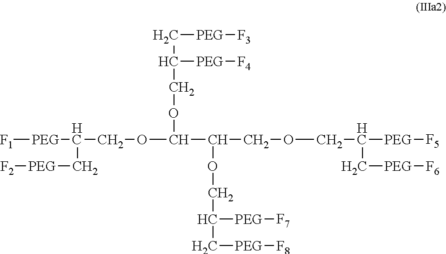
in formula IIIa2, F₁-F₈ are the same or not the same —Z—Y type structures,
Z₁-Z₈ are linking groups, and
Y₁-Y₈ are terminal active groups; 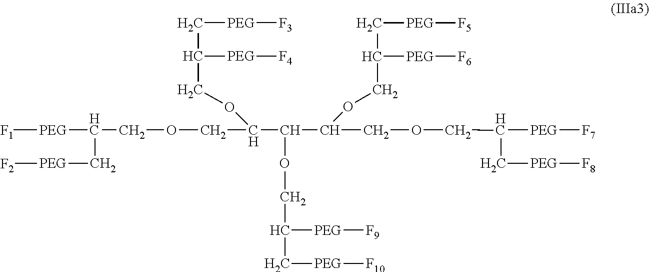
in formula IIIa3, F₁-F₁₀ are the same or not the same —Z—Y type structures,
Z₁-Z₁₀ are linking groups, and
Y₁-Y₁₀ are terminal active groups; 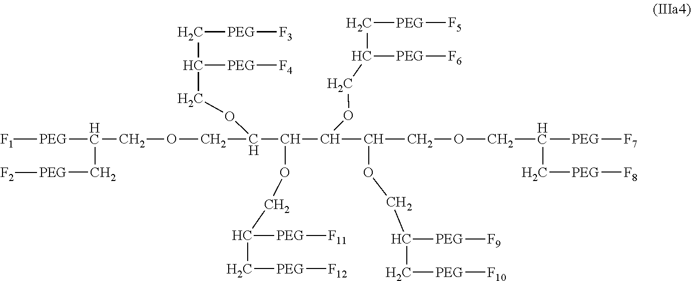
in formula IIIa4, F₁-F₁₂ are the same or not the same —Z—Y type structures,
Z₁-Z₁₂ are linking groups, and
Y₁-Y₁₂ are terminal active groups; 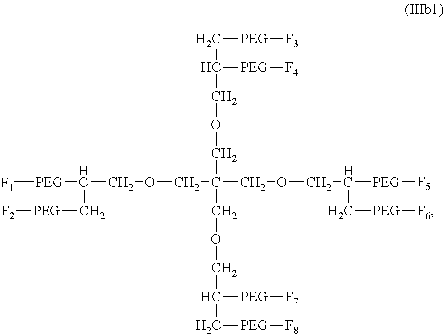
in formula IIIb1, F₁-F₈ are the same or not the same —Z—Y type structures,
Z₁-Z₈ are linking groups, and
Y₁-Y₈ are terminal active groups; 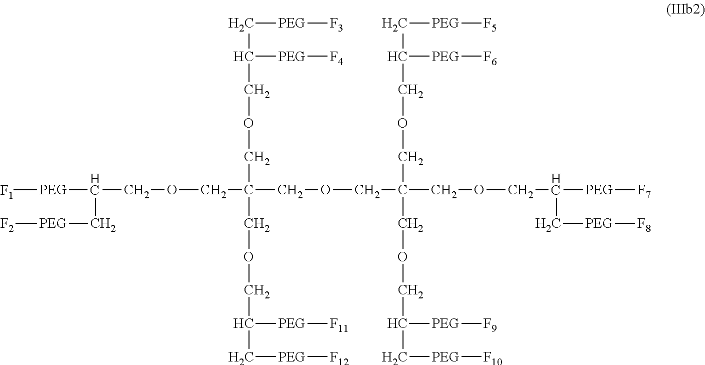
in formula IIIb2, F₁-F₁₂ are the same or not the same —Z—Y type structures,
Z₁-Z₁₂ are linking groups, and
Y₁-Y₁₂ are terminal active group;
and 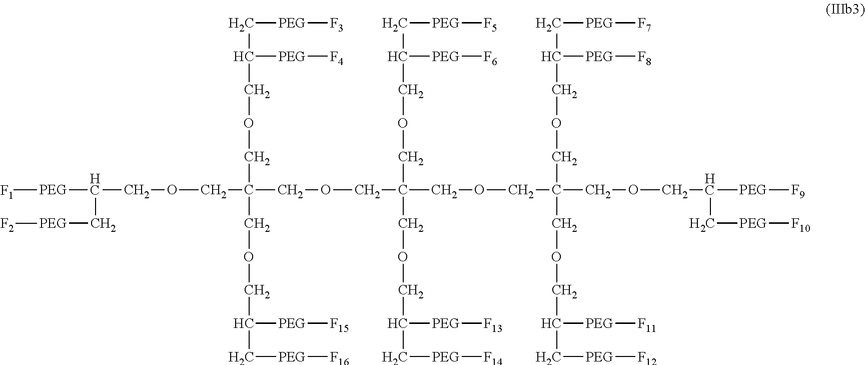
in formula IIIb3, F₁-F₁₆ are the same or not the same —Z—Y type structures,
Z₁-Z₁₆ are linking groups, and
Y₁-Y₁₆ are terminal active groups.

9. The multi-armed polyethylene glycol active derivative of claim 8, wherein the linking group is selected from the group consisting of —O(CH₂)ᵢ—, —O(CH₂)ᵢNH—, —O(CH₂)ᵢOCOO—, —O(CH₂)ᵢOCONH—, —O(CH₂)ᵢNHCOO—, —O(CH₂)ᵢNHCONH—, —OCO(CH₂)ᵢCOO—, —O(CH₂)ᵢCOO—, —O(CH₂)ᵢCONH— and —O(CH₂)ᵢNHCO(CH₂)ₑ—; i is an integer between 0 and 10, and e is an integer between 1 and 10;
the terminal active group is selected from the group consisting of —H, —NH₂, —COCH═CH₂, —COC(CH₃)═CH₂, 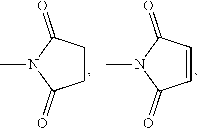
—SH, 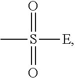
—CHO, —C≡CH, —PO₃H, —N₃, —CN, —CH═CH₂, 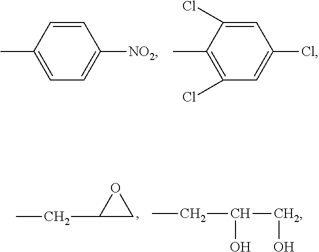
—CH═CH—COOH, —N═C═O, 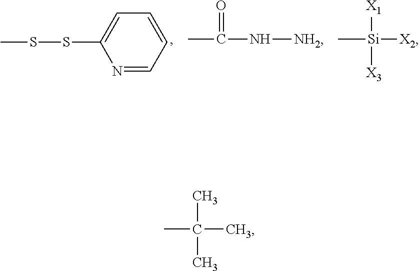
C1-6 alkyl and C1-6 alkoxy;
E is a C1-10 hydrocarbyl or a fluorine atom-containing C1-10 hydrocarbyl; and
X₁, X₂ and X₃ are the same or not the same C1-10 hydrocarbyl or C1-6 alkoxy.

10. The multi-armed polyethylene glycol active derivative of claim 9, wherein E is selected from the group consisting of methyl, ethyl, propyl, butyl, vinyl, phenyl, benzyl, p-methylphenyl, and trifluoromethyl; and
X₁, X₂ and X₃ are independently selected from the group consisting of methyl, ethyl, propyl, isopropyl, phenyl, benzyl, methoxy, and ethoxy.

11. The multi-armed polyethylene glycol active derivative of claim 1, wherein the multi-armed polyethylene glycol active derivative has a structure of formula IIIa1-a1: 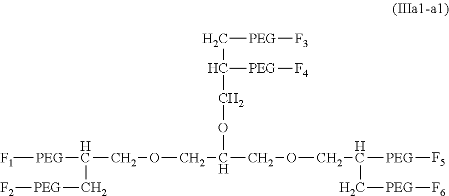
wherein, F₁ is a —Z—Y type structure different from F₂, F₃, F₄, F₅ and F₆;
F₁ is a —Z₁—Y₁ type structure;
F₂, F₃, F₄, F₅ and F₆ are —Z₂—Y₂ type structures;
Z₁ is a linking group selected from the group consisting of —O(CH₂)ᵢOCOO—, —O(CH₂)ᵢOCONH—, —OCO(CH₂)ᵢCOO—, —O(CH₂)ᵢCOO— and —O(CH₂)ᵢCONH—; i is an integer between 0 and 10;
Z₂ is a linking group selected from the group consisting of —O(CH₂)_{i′}—, —O(CH₂)_{i′}NH—, —O(CH₂)_{i′}NHCOO—, —O(CH₂)_{i′}NHCONH— and —O(CH₂)_{i′}NHCO(CH₂)ₑ—; i′ is an integer between 0 and 10; and e is an integer between 1 and 10;
Y is a terminal active group selected from the group consisting of —H, —NH₂, —COCH═CH₂, —COC(CH₃)═CH₂, 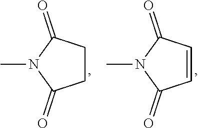
—SH, 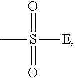
—CHO, —C≡CH, —PO₃H, —N₃, —CN, —CH═CH₂, 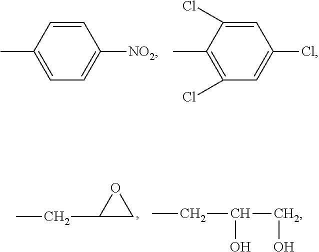
—CH═CH—COOH, —N═C═O, 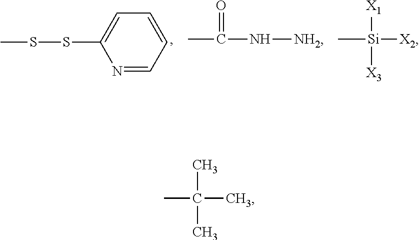
C1-6 alkyl and C1-6 alkoxy;
E is a C1-10 hydrocarbyl or a fluorine atom-containing C1-10 hydrocarbyl;
X₁, X₂ and X₃ are the same or not the same C1-10 hydrocarbyl or C1-6 alkoxy; and
PEG is the same or not the same —(OCH₂CH₂)ₘ—, and the average value of m is an integer between 3 and 250.

12. The multi-armed polyethylene glycol active derivative of claim 1, wherein the multi-armed polyethylene glycol active derivative has a structure of formula IIIb1-a1: 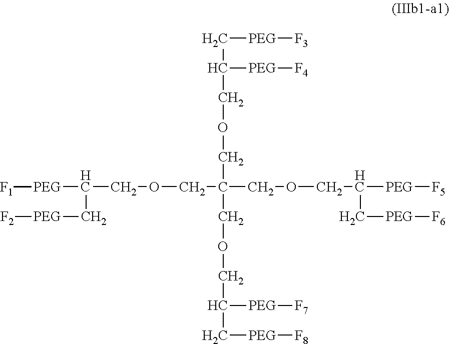
wherein, F₁ is a —Z—Y type structure different from F₂, F₃, F₄, F₅, F₆, F₇ and F₈;
F₁ is a —Z₁—Y₁ type structure;
F₂, F₃, F₄, F₅, F₆, F₇ and F₈ are —Z₂—Y₂ type structures;
Z₁ is a linking group selected from the group consisting of —O(CH₂)ᵢOCOO—, —O(CH₂)ᵢOCONH—, —OCO(CH₂)ᵢCOO—, —O(CH₂)COO— and —O(CH₂)CONH—; i is an integer between 0 and 10;
Z₂ is a linking group selected from the group consisting of —O(CH₂)_{i′}—, —O(CH₂)_{i′}NH—, —O(CH₂)_{i′}NHCOO—, —O(CH₂)_{i′}NHCONH— and —O(CH₂)_{i′}NHCO(CH₂)ₑ—; i′ is an integer between 0 and 10; and e is an integer between 1 and 10;
Y is a terminal active group selected from the group consisting of —H, —NH₂, —COCH═CH₂, —COC(CH₃)═CH₂, 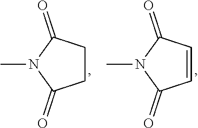
—SH, 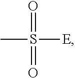
—CHO, —C≡CH, —PO₃H, —N₃, —CN, —CH═CH₂, 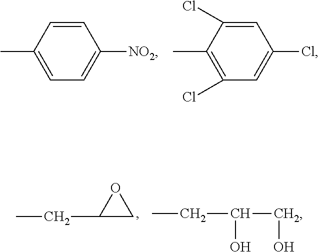
—CH═CH—COOH, —N═C═O, 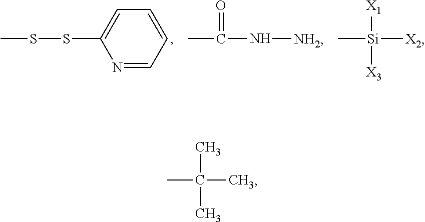
C1-6 alkyl and C1-6 alkoxy;
E is a C1-10 hydrocarbyl or a fluorine atom-containing C1-10 hydrocarbyl;
X₁, X₂ and X₃ are the same or not the same C1-10 hydrocarbyl or C1-6 alkoxy; and
PEG is the same or not the same —(OCH₂CH₂)ₘ—, and the average value of m is an integer between 3 and 250.

13. The multi-armed polyethylene glycol active derivative of claim 1, wherein the multi-armed polyethylene glycol active derivative has a structure of formula IIIb1-a2: 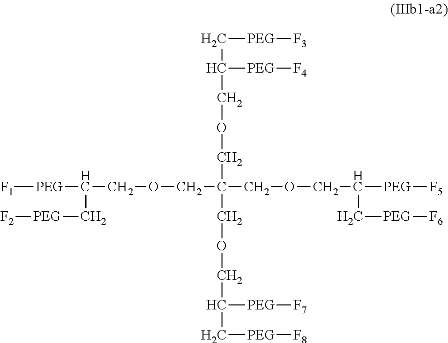
wherein, F₁ and F₂ are —Z—Y type structures different from F₃, F₄, F₅, F₆, F₇, and F₈;
F₁ and F₂ are —Z₁—Y₁ type structures;
F₃, F₄, F₅, F₆, F₇ and F₈ are —Z₂—Y₂ type structures;
Z₁ is a linking group selected from the group consisting of —O(CH₂)ᵢOCOO—, —O(CH₂)ᵢOCONH—, —OCO(CH₂)ᵢCOO—, —O(CH₂)ᵢCOO— and —O(CH₂)ᵢCONH—; i is an integer between 0 and 10;
Z₂ is a linking group selected from the group consisting of —O(CH₂)_{i′}—, —O(CH₂)_{i′}NH—, —O(CH₂)_{i′}NHCOO—, —O(CH₂)_{i′}NHCONH— and —O(CH₂)_{i′}NHCO(CH₂)ₑ—; i′ is an integer between 0 and 10; and e is an integer between 1 and 10;
Y is a terminal active group selected from the group consisting of —H, —NH₂, —COCH═CH₂, —COC(CH₃)═CH₂, 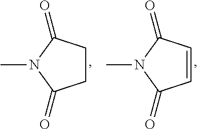
—SH, 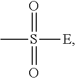
—CHO, —C≡CH, —PO₃H, —N₃, —CN, —CH═CH₂, 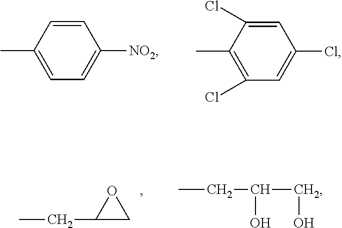
—CH═CH—COOH, —N═C═O, 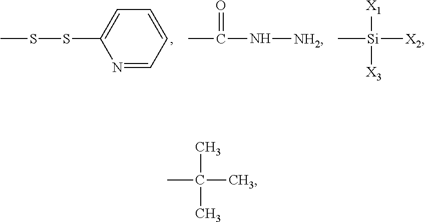
C1-6 alkyl and C1-6 alkoxy;
E is a C1-10 hydrocarbyl or a fluorine atom-containing C1-10 hydrocarbyl;
X₁, X₂ and X₃ are the same or not the same C1-10 hydrocarbyl or C1-6 alkoxy; and
PEG is the same or not the same —(OCH₂CH₂)ₘ—, and the average value of m is an integer between 3 and 250.

14. The multi-armed polyethylene glycol active derivative of claim 1, wherein the multi-armed polyethylene glycol active derivative has structures of III-1 to III-11: 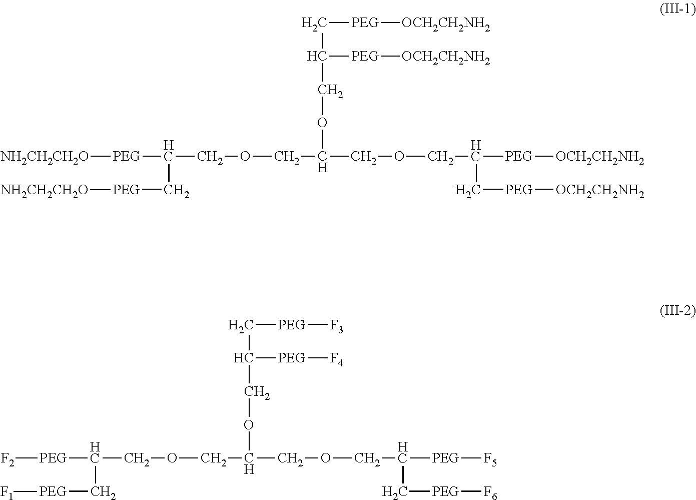
in formula III-2, F₁, F₂, F₃, F₄, F₅ and F₆ are all 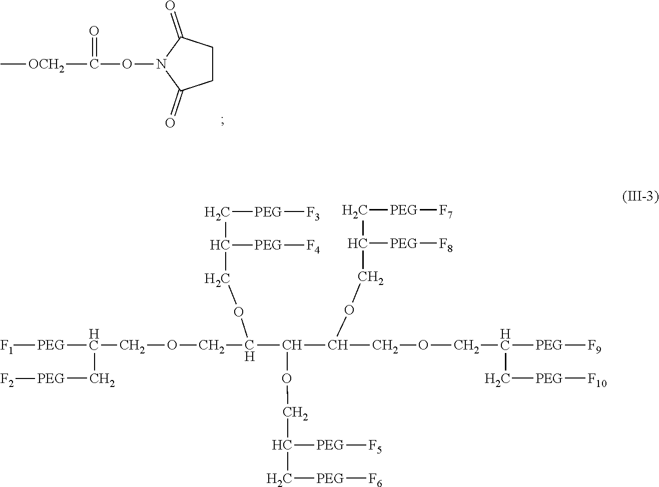
in formula III-3, F₁, F₂, F₃, F₄, F₅, F₆, F₇, F₈, F₉ and F₁₀ are all 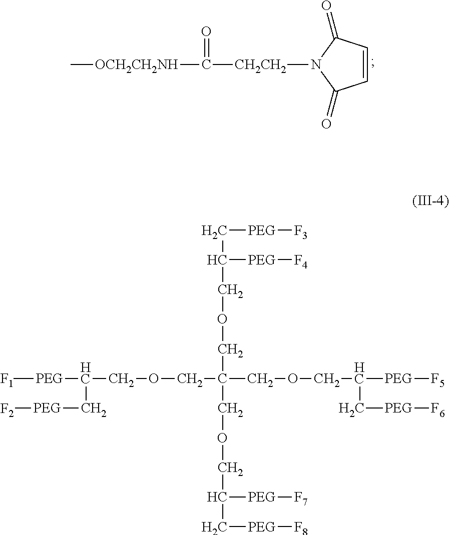
in formula III-4, F₁, F₂, F₃, F₄, F₅, F₆, F₇ and F₈ are all 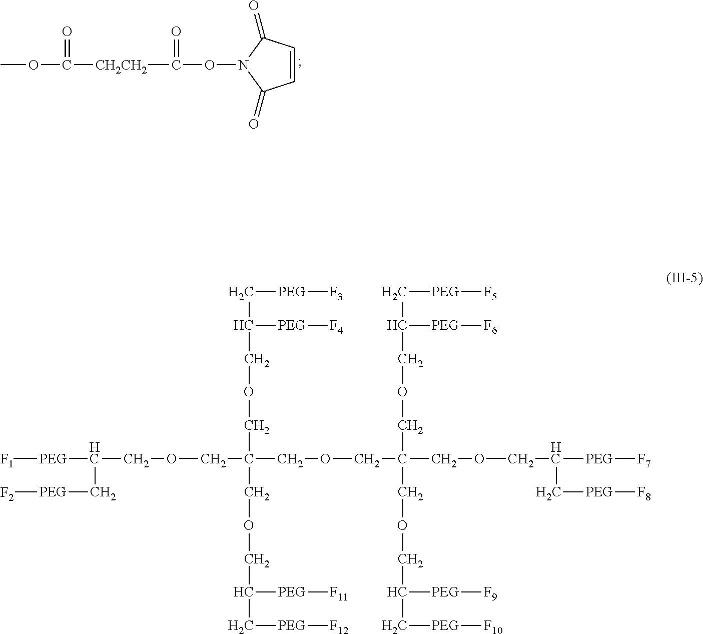
in formula III-5, F₁, F₂, F₃, F₄, F₅, F₆, F₇, F₈, F₉, F₁₀, F₁₁ and F₁₂ are all 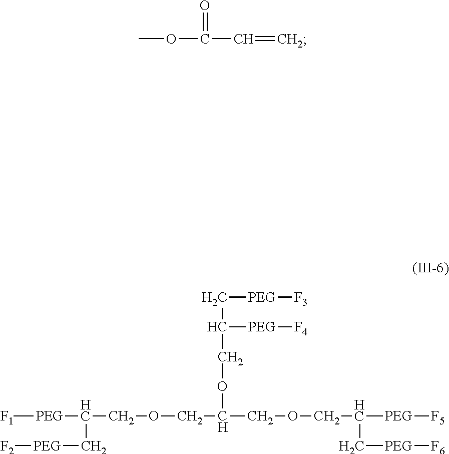
in formula III-6, F₁ is —OCH₂COOH, and F₂, F₃, F₄, F₅ and F₆ are all —OH; 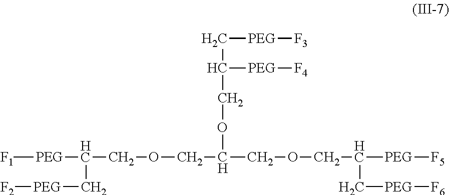
in formula III-7, F₁ is —OCH₂COOH, and F₂, F₃, F₄, F₅ and F₆ are all —OCH₂CH₂—NH₂; 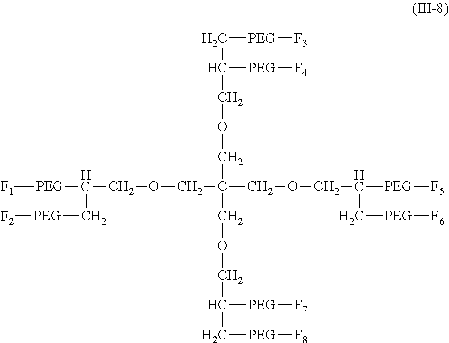
in formula III-8, F₁ is —OCH₂COOH, and F₂, F₃, F₄, F₅, F₆, F₇ and F₈ are all —OH; 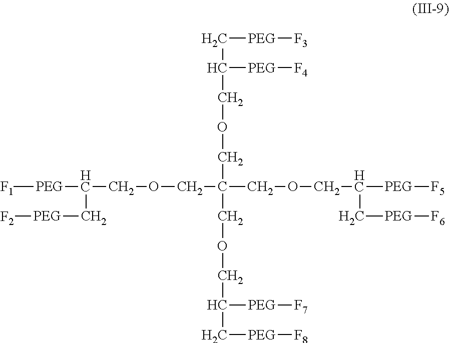
in formula III-9, F₁ and F₂ are —OCH₂COOH, and F₃, F₄, F₅, F₆, F₇ and F₈ are all —OH; 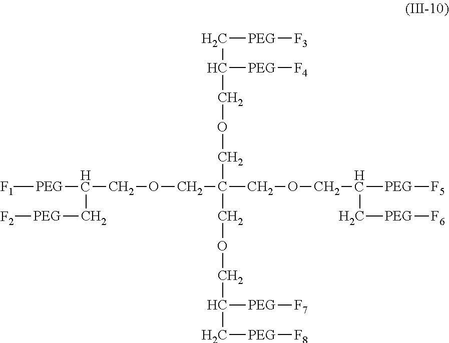
in formula III-10, F₁ is —OCH₂COOH, and F₂, F₃, F₄, F₅, F₆, F₇ and F₈ are all —OCH₂CH₂—NH₂;
and 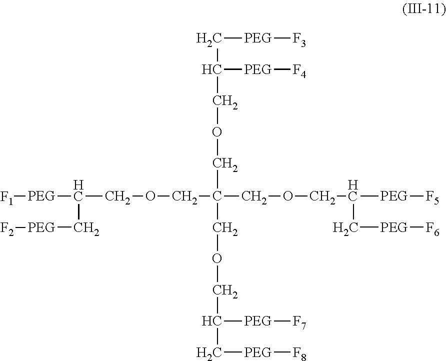
in formula III-11, F₁ is: 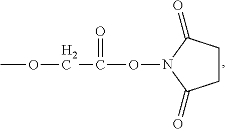
and F₂, F₃, F₄, F₅, F₆, F₇ and F₈ are all:

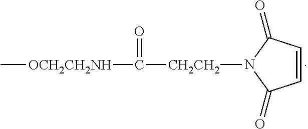

15. The multi-armed polyethylene glycol active derivative of claim 14, wherein the multi-armed polyethylene glycol active derivative has a number average molecular weight of 1,500 to 80,000.

16. The multi-armed polyethylene glycol active derivative of claim 14, wherein the multi-armed polyethylene glycol active derivative has a number average molecular weight of 10,000 to 50,000.

17. The multi-armed polyethylene glycol active derivative of claim 14, wherein the multi-armed polyethylene glycol active derivative has a number average molecular weight of 10,000 to 30,000.

18. A conjugate of the multi-armed polyethylene glycol active derivative of claim 1 and a drug molecule.

19. The conjugate of claim 18, wherein the drug molecule is inonotecan or docetaxel.

20. A gel formed by the multi-armed polyethylene glycol active derivative of claim 1.
